(19) United States
(12) Patent Application Publication
(10) Pub. No.: US 2009/0111810 A1
(43) Pub. Date: Apr. 30, 2009

(54) SUBSTITUTED PYRIMIDINE-5-CARBOXAMIDE AND 5-CARBOXYLIC ESTER KINASE INHIBITORS

(76) Inventors: Peter J. Connolly, New Providence, NJ (US); Terry V. Hughes, Blue Bell, PA (US); Steven K. Wetter, Flemington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/252,718

(22) Filed: Oct. 16, 2008

Related U.S. Application Data
(60) Provisional application No. 60/981,946, filed on Oct. 23, 2007.

(51) Int. Cl. A61K 31/5377; C07D 401/12; C07D 413/12; A61P 25/00; A61K 31/506

(52) U.S. Cl. 514/235.8; 544/329; 514/256; 544/122

(57) ABSTRACT

The present invention is directed to substituted pyrimidine-5-carboxamide and 5-carboxylic ester compounds of Formula (I):

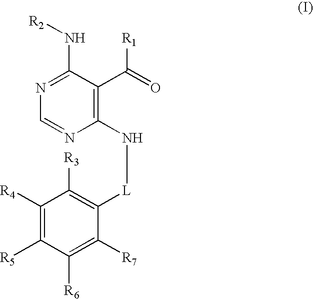

and forms thereof wherein R₁, R₂, R₃, R₄, R₅, R₆, R₇ and L are as defined herein and their synthesis and use as kinase inhibitors for treating a chronic or acute protein kinase mediated disease.

### FIELD OF THE INVENTION

The present invention is directed to substituted pyrimidine-5-carboxamide and 5-carboxylic ester compounds and forms thereof and methods of preparation and use thereof as kinase inhibitors.

### BACKGROUND OF TUE INVENTION

In general, protein kinases are the largest set of structurally related phosphoryl transferases, have highly conserved structures and catalytic functions and may be categorized into families by the substrates they phosphorylate (e.g., protein-tyrosine, protein-serine/threonine, histidine and the like) and are responsible for the control of a wide variety of cellular signal transduction processes.

Examples of protein-tyrosine kinases include, but are not limited to, Irk, IGFR-1, Zap-70, Bmx, Btk, CHK (Csk homologous kinase), CSK (C-terminal Src Kinase), Itk-t Src (c-Src, Lyn, Fyn, Lek, Syk, Hck, Yes, Blk, Fgr and Frk), Tec, Txk/Rlk, Abl, ECFR (EGFR-1/ErbB-1, ErbB-2/NEU/HER-2, ErhB-3 and ErbB-4), FAK, FGF1R (also FUFR1 or FUR-1), FGF2R (also FGR-2), MET (also Met-1 or c-MET), PDGFR (α and β), Tie-1, Tie-2 (also Tek-1 or Tek), VEGFR1 (also FLT-1), VEGFR2 (also KDR), FLT-3, FLT-4, c-KIT, JAK1, JAK2, JAK3, TYK2, LOK, RET, TRA, PYK2, ALK (Anaplastic Lymphoma Kinase), EPHA (1-8), EPHB (1-6), RON, Fes, Fer or EPHB4 (also EPHB4-1).

Examples of protein-serine/threonine kinases include, but are not limited to, Ark, ATM (1-3), CamK (I-IV), CamKK, Chk1 and 2 (Checkpoint kinases), CK1, CK2, Erk, IKK-1 (also IKK-ALPHA or CHUK), IKK-2 (also IKK-BETA), Ilk, Jnk (1-3), LimK (1 and 2), MLK3Raf (A, B, and C), CDK (1-10), PKC (including all PKC subtypes), Plk (1-3), NIK, Pak (1-3), PDK1, PKR, RhoK, RIP, RIP-2, GSK3 (α and β). PKA, P38, Erk (1-3), PKB (including all PKB subtypes) (also AKT-1, AKT-2, AKT-3 or AKT3-1), IRAK1, FRK, SGK, TAK1 or Tpl-2 (also COT).

Protein kinases play very important roles in the normal regulation of cell growth. However, as a result of dysregulation of the tyrosine kinases (receptor or non-receptor) or the ligands of the receptor tyrosine kinases, signaling can become deregulated, resulting in uncontrolled cell proliferation leading to cancer or a related disease, disorder or syndrome.

Protein kinases catalyze and regulate the process of phosphorylation, whereby the kinases covalently attach phosphate groups to proteins or lipid targets in response to a variety of extracellular signals: hormones, neurotransmitters, growth and differentiation factors, cell cycle events, environmental stresses, nutritional stresses and the like.

Phosphorylation modulates or regulates a variety of cellular processes such as proliferation, growth, differentiation, metabolism, apoptosis, motility, transcription, translation and other signaling processes. Defective control of protein phosphorylation due to unregulated cellular mitosis, unregulated cell proliferation and upregulated kinase activity has been implicated in a number of diseases and disease conditions, such as osteoarthritis, rheumatoid arthritis, synovial pannus invasion in arthritis, multiple sclerosis, myasthenia gravis, diabetes mellitus, diabetic angiopathy, diabetic retinopathy, retinal vessel proliferation, inflammatory bowel disease, Crohn's disease, ulcerative colitis, bone diseases, transplant or bone marrow transplant rejection, lupus, chronic pancreatitis, cachexia, septic shock, fibroproliferative and differentiative skin diseases or disorders, central nervous system diseases, neurodegenerative diseases, disorders or conditions related to nerve damage and axon degeneration subsequent to a brain or spinal cord injury, acute or chronic cancer, ocular diseases, viral infections, heart disease, lung or pulmonary diseases or kidney or renal diseases. Therefore, kinase inhibitors have potential use as therapeutic agents.

The term “myasthenia gravis” means a disease having the characteristic feature of easy fatigue of certain voluntary muscle groups on repeated use. Muscles of the face or upper trunk are especially likely to be affected. In most and perhaps all cases, the disease is due to the development of autoantibodies against the acetylcholine receptor in neuromuscular junctions. Immunization of animals with this receptor protein leads to a disease with the features of myasthenia gravis.

In reference to “synovial pannus invasion in arthritis)” the term “pannus” means a disease whereby vascularised granulation tissue rich in fibroblasts, lymphocytes and macrophages, derived from synovial tissue, overgrows the bearing surface of the joint in rheumatoid arthritis and is associated with the breakdown of the articular surface.

The tyrosine kinases can further be categorized by whether they are receptor tyrosine kinases or non-receptor tyrosine kinases. The receptor tyrosine kinases span the cell membrane with a ligand interacting domain protruding from the cell, with a hydrophobic trans-membrane domain, and a cytoplasmic domain that contains the catalytic kinase domain and other regulatory sequences. Non-receptor tyrosine kinases are often myristylated or modified by the addition of other hydrophobic moieties that allow them to be anchored to the cell membrane.

The epidermal growth factor receptor (EGFR) tyrosine-kinase family includes the receptors EGFR (also referred to as EGFR-1 or Erb-B1), HER-2 (or neu), EGFR3 and EGFR4. Epidermal Growth Factor (EGF), Transforming Growth Factor-α (TGF-α) and the HER-2 ligand heregulin are three of the ligands that bind to the EGFR receptors.

For example, EGFR overexpression or mutation of one or more EGFR kinase family members has been commonly involved in cancer and other diseases characterized by uncontrolled or abnormal cell growth. Deregulation of EGFR has also been associated with epidermoid tumors, head and neck tumors, breast tumors and tumors involving other major organs, such as the lungs and gastrointestinal tract. The clinically prevalent cancers related to EGFR include lung, gastric and head and neck cancer (Klijn J G, Berns P M, Schmitz P I and Foekens J A; The clinical significance of epidermal growth factor receptor (EGF-R) in human breast cancer: a review on 5232 patients, Endocr. Rev., 1992, 13, 3-17; Salomon D and Gullick W; The erbb family of receptors and their ligands: Multiple targets for therapy, Signal, 2001, 2, 4-11).

In treating cancers of the head such as brain cancers and the like, the ability of small molecule EGFR inhibitors to penetrate the blood brain barrier could have therapeutic advantages since EGFR is often overexpressed in primary brain tumors and also in breast and non-small cell lung carcinomas that frequently metastasize to the brain (Eckstrand A J, Sugawa N, James C D and Collins V P; Amplified and rearranged epidermal growth factor receptor genes in human glioblastomas reveal deletions of sequences encoding portions of the N- and/or C-terminal tails, Proc. Acad. Natl. Sci. USA, 1992, 89, 4309-4313; and, Wickstrand C J, Hale L P, Batra S K, Hill M L, Humphrey P A, Kurpad S N, McLendon R E, Moscatello X, Pegram C N, Reist C J, Traweek S T, Wong A J, Zalutsky M R and Bigner, D D; Monoclonal antibodies against EGFRvIII are tumor specific and react with breast and lung carcinomas and malignant gliomas, Cancer Res., 1995, 55, 3140-3148).

Diseases associated with increased EGFR expression include proliferative glomerulonephritis, diabetes-induced renal disease and chronic pancreatitis.

EGFR inhibitors tested in neurite outgrowth assays have activity in promoting neurite outgrowth in both cerebellar granule cells and dorsal root ganglion neurons, likely by acting directly on neurons to block neuronal inhibitory responses to myelin inhibitors, and thus an EGFR inhibitor may have potential use for promoting axon regeneration after brain and spinal cord injury (V. Koprivica, et al, EGFR activation mediates inhibition of axon regeneration by myelin and chondroitin sulfate proteoglycans, Science, 2005, 310, 106).

HER1 and HER2 overexpression has been implicated in a variety of cancers, such as bladder, breast, colorectal, endometrial, esophageal, gastric (stomach), glioma head and neck, lung (non-small cell lung cancer), ovarian, pancreatic, renal and prostate cancer.

Comparing the overexpression of HER1 and HER2 in tumors, according to order of prevalence, HER1 overexpression is found in breast, renal cell, lung, colorectal, head and neck, ovarian, pancreatic, glioma, bladder, esophageal, gastric, endometrial and cervical cancer tumors; in contrast, HER2 overexpression is found in esophageal, head and neck, lung, gastric, renal cell, breast, bladder, ovarian and colorectal, prostate and endometrial cancer tumors (Horizons in Cancer Therapeutics: From Bench to Bedside, Signal Transduction Inhibitors, 2001, 2(2), ISSN 1532-3048).

While the degree of HER2 overexpression in breast and ovarian cancer is not as great as in some other cancers, HER2 has been found to be responsible for these clinically prevalent cancers (Slamon D J, Clark G M, Wong S G, Levin W J, Ullrich A and McGuire W L; Human breast cancer: Correlation of relapse and survival with amplification of HER-2/neu oncogene, Science, 1987, 235, 177-82; Slamon D J, Godolphin W, Jones L A, Holt J A, Wong S G, Keith D E, et al; Studies of the HER-2/neu proto-oncogene in human breast and ovarian cancer, Science, 1989, 244, 707-712; Hetzel D J, Wilson T O, Keeney G L, Roche P C, Cha S S and Podrantz K C; HER-2/neu expression: A major prognostic factor in endometrial cancer, Gynecol. Oncol., 1992, 47, 179-85).

Furthermore, patients with HER-2 overexpressing breast cancer frequently experience metastases to the brain (Kirsch D C and Hochberg F H; Targeting HER-2 in brain metastases from breast cancer, Clin. Can. Res., 2003, 9, 5435-5436). These patients have an extremely poor prognosis and intracerebral tumors are often the cause of death. Autopsy revealed that 20-30% of patients who die of breast cancer have brain metastases (Grossi P M, Ochiai H, Archer G E, McLendon R E, Zalutsky M R, Friedman A H, Friedman H S, Bigner D D and Sampson J H; Efficacy of intracerebral microinfusion of trastuzumab in an athymic rat model of intracerebral metastatic breast cancer, Clin. Can. Res., 2003, 9, 5514-5520).

Human cytomegalovirus (CMV) is a widespread opportunistic human herpes virus that causes severe and fatal diseases in those who are immune compromised and in transplant recipients. CMV is also a leading cause of atherosclerosis and virally mediated birth defects. The human CMV uses the EGFR receptor to enter cells during infection, ECFR is autophosphorylated and the downstream signal transduction pathway components are activated; however, the EGFR specific inhibitor tyrphostin AG1478 has been shown to reduce the viral load in cells that were infected in the presence of the tyrphostin (Wang X, et al., Nature, 24 Jul. 2003, Vol. 424, 456-461). Accordingly, potent EGFR selective inhibitors may be useful in anti-CMV therapy.

Certain oxime substituted pyrimidines are registered by the Chemical Abstracts Society (CAS) such as 4,6-diamino-5-pyrimidinecarboxaldehyde oxime (CAS Registry No.: 109831-69-8) and N,N′-dimethyl-5-[(methylimino)methyl]-4,6-pyrimidinediamine (CAS Registry No.: 14160-97-5) and described in Hererocycles, 1987, 25(1), 343-5. Certain references describe substituted pyrimidine compounds such as United States patents. U.S. Pat. No. 6,080,750, U.S. Pat. No. 6,107,301 and U.S. Pat. No. 6,833,378.

There is a need for potent small-molecule kinase inhibitors of one or more of the EGFR, HER-2 kinase proteins and the like possessing anti-tumor cell proliferation activity, and as such are useful for treating or ameliorating a EGFR or HER-2 kinase receptor mediated, angiogenesis-mediated or hyperproliferative disorder.

### SUMMARY OF THE INVENTION

The present invention is directed to a compound of Formula (I):

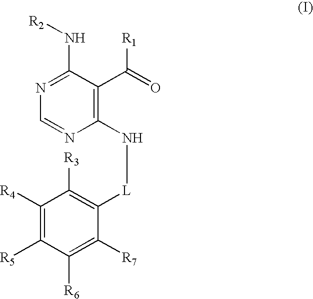

and forms thereof, wherein R₁, R₂, R₃, R₄, R₅, R₆, R₇ and L are as defined herein.

An example of the present invention includes a compound of Formula (I) and forms thereof as a protein kinase inhibitor.

An example of the present invention includes use of a compound of Formula (I) and forms thereof as an inhibitor of a protein kinase such as EGFR, HER-2 and the like comprising contacting the protein kinase domain or receptor with the compound.

The present invention is further directed to a method for treating, preventing or ameliorating a chronic or acute protein kinase mediated disease, disorder or condition in a subject in need thereof comprising administering to the subject an effective amount of a compound of Formula (I) and forms thereof.

These and other aspects and advantages of the invention, which will become apparent in light of the detailed description below, are achieved through use of the compounds of this invention.

### DETAILED DESCRIPTION OF THE INVENTION

The present invention is directed to a compound of Formula (I):

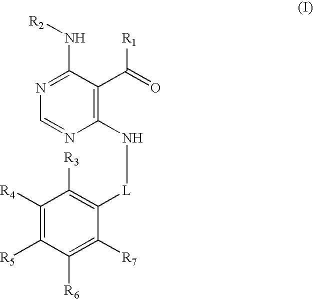

and forms thereof, wherein
L is selected from the group consisting of a bond, C₁₋₆alkyl and halo-C₁₋₆alkyl;
R₁ is selected from the group consisting of hydrogen, hydroxy, C₁₋₈alkoxy, hydroxy-C₁₋₈alkoxy, amino, C₁₋₈alkyl-amino, C₁₋₈alkoxy-C₁₋₈alkyl-amino, amino-amino, C₁₋₈alkyl-amino-amino, C₁₋₈alkyl-carbonyl-amino-amino, aryl-C₁₋₈alkoxy, aryl-amino, aryl-C₁₋₈alkyl-amino, heterocyclyl-C₁₋₈alkoxy, heterocyclyl-C₁₋₈alkyl-amino, heterocyclyl-C₁₋₈alkyl-carbonyl-amino, heterocyclyl-C₁₋₈alkyl-carbonyl-amino-amino, heterocyclyl and benzofused-heterocyclyl,
wherein each instance of aryl is optionally substituted with one, two, three, four or five substituents each selected from the group consisting of halogen, hydroxy, C₁₋₈alkyl, C₁₋₈alkoxy, amino, C₁₋₈alkyl-amino, C₁₋₈alkyl-carbonyl and C₁₋₈alkoxy-carbonyl, and
wherein each instance of heterocyclyl is optionally substituted with one, two, three or four substituents each selected from the group consisting of hydroxy, C₁₋₈alkyl, C₁₋₈alkoxy, amino, C₁₋₈alkyl-amino, C₁₋₈alkyl-carbonyl and C₁₋₈alkoxy-carbonyl;
R₂ is selected from the group consisting of hydrogen, C₁₋₈alkyl and C₁₋₈alkoxy; and
R₃, R₄, R₅, R₆ and R₇ are each selected from the group consisting of hydrogen, halogen, hydroxy, cyano, nitro, C₁₋₈alkyl, C₁₋₈alkoxy, C₁₋₈alkoxy-C₁₋₈alkyl, hydroxy-C₁₋₈alkyl, halo-C₁₋₈alkyl, hydroxy-C₁₋₈alkoxy, halo-C₁₋₈alkoxy, cyano-C₁₋₈alkyl, amino, C₁₋₈alkyl-amino, amino-C₁₋₈alkyl, C₁₋₈alkyl-amino-C₁₋₈alkyl, C₁₋₈alkyl-amino-C₁₋₈alkyl-amino-carbonyl, C₁₋₈alkoxy-imino-C₁₋₈alkyl, C₁₋₈alkoxy-imino-(aryl)C₁₋₈alkyl, carboxy, C₁₋₈acyl, C₁₋₈acyl-amino, C₁₋₈alkyl-carbonyl, C₁₋₈alkoxy-carbonyl, thio-C₁₋₈alkyl, substituted phosphonic acid, C₃₋₁₂cycloalkyl, aryl, aryloxy, aryl-amino, aryl-C₁₋₈alkyl, aryl-C₁₋₈alkoxy, aryl-carbonyl-C₁₋₈alkyl, aryl-amido, heteroaryl, heteroaryloxy, heteroaryl-C₁₋₈alkoxy, heteroaryl-amino-sulfonyl, heterocyclyl and benzofused-heterocyclyl,
wherein phosphonic acid is substituted on the phosphorous atom with two substituents selected from the group consisting of hydroxy and C₁₋₈alkoxy,
wherein each instance of heterocyclyl and benzofused-heterocyclyl is optionally substituted on one or two heterocyclyl carbon atoms with an oxo substituent, and
wherein each instance of heteroaryl is optionally substituted with one, two, three, four or five substituents each selected from the group consisting of C₁₋₈alkyl, amino-C₁₋₈alkyl, C₁₋₈alkyl-amino-C₁₋₈alkyl, carboxy, C₁₋₈acyl and C₁₋₈alkoxy-carbonyl.

An example of a compound of Formula (I) includes a compound and forms thereof, wherein
L is a bond;
R₁ is selected from the group consisting of hydrogen, hydroxy, C₁₋₈alkoxy, C₁₋₈alkyl-amino, C₁₋₈alkoxy-C₁₋₈alkyl-amino, amino-amino, aryl-C₁₋₈alkoxy, aryl-amino, aryl-C₁₋₈alkyl-amino, heterocyclyl-C₁₋₈alkoxy, heterocyclyl-C₁₋₈alkyl-amino, and heterocyclyl-C₁₋₈alkyl-carbonyl-amino-amino,
wherein each instance of aryl is optionally substituted with one substituent selected from the group consisting of halogen and C₁₋₈alkoxy;
R₂ is hydrogen; and
R₃, R₄, R₅, R₆ and R₇ are each selected from the group consisting of hydrogen, halogen, and aryl-C₁₋₈alkoxy.

An example of a compound of Formula (I) includes a compound and forms thereof, wherein
R₁ is selected from the group consisting of hydrogen, hydroxy, C₁₋₈alkoxy, C₁₋₈alkyl-amino, C₁₋₈alkoxy-C₁₋₈alkyl-amino, amino-amino, phenyl-C₁₋₈alkoxy, phenyl-amino, phenyl-C₁₋₈alkyl-amino, morpholinyl-C₁₋₈alkoxy, piperidinyl-C₁₋₈alkyl-amino, morpholinyl-C₁₋₈alkyl-amino and morpholinyl-C₁₋₈alkyl-carbonyl-amino-amino,
wherein each instance of phenyl is optionally substituted with one substituent selected from the group consisting of halogen and C₁₋₈alkoxy.

An example of a compound of Formula (I) includes a compound and forms thereof wherein R₃, R₄, R₅, R₆ and R₇ are each selected from the group consisting of hydrogen, halogen and phenyl-C₁₋₈alkoxy.

An example of a compound of Formula (I) includes a compound and forms thereof wherein R₄ and R₅, respectively, are halogen and phenyl-C₁₋₈alkoxy.

An example of a compound of Formula (I) includes a compound and forms thereof, wherein R₄ and R₅, respectively, are chloro and phenyl-C₁₋₅alkoxy.

The present invention is further directed to a compound of Formula (La):

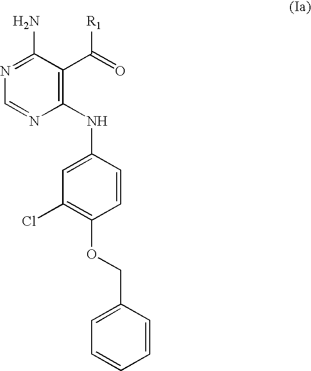

and forms thereof, wherein
R₁ is selected from the group consisting of hydrogen, hydroxy, C₁₋₈alkoxy, C₁₋₈alkyl-amino, C₈alkoxy-C₁alkyl-amino, amino-amino, aryl-C₁₋₈alkoxy, aryl-amino, aryl-C₁₋₈alkyl-amino, heterocyclyl-C₁₋₈alkoxy, heterocyclyl-C₁₋₈alkyl-amino, and heterocyclyl-C₁₋₈alkyl-carbonyl-amino-amino,
wherein each instance of aryl is optionally substituted with one substituent selected from the group consisting of halogen and C₁₋₈alkoxy.

An example of a compound of Formula (Ia) includes a compound and forms thereof, wherein
R₁ is selected from the group consisting of hydrogen, hydroxy, C₁₋₈alkoxy, amino-amino, aryl-amino, heterocyclyl-C₁₋₈alkoxy, heterocyclyl-C₁₋₈alkyl-amino, and heterocyclyl-C₁₋₈alkyl-carbonyl-amino-amino,
wherein aryl is optionally substituted with one substituent selected from the group consisting of halogen and C₁₋₈alkoxy.

An example of a compound of Formula (I) includes a compound and forms thereof, wherein
R₁ is selected from the group consisting of hydrogen, hydroxy, C₁₋₈alkoxy, C₁₋₈alkyl-amino, C₁₋₈alkoxy-C₁₋₈alkyl-amino, amino-amino, phenyl-C₁₋₈alkoxy, phenyl-amino, phenyl-C₁₋₈alkyl-amino, morpholinyl-C₁₋₈alkoxy, piperidinyl-C₁₋₈alkyl-amino, morpholinyl-C₁₋₈alkyl-amino and morpholinyl-C₁₋₈alkyl-carbonyl-amino-amino,
wherein each instance of phenyl is optionally substituted with one substituent selected from the group consisting of halogen and C₁₋₈alkoxy.

An example of a compound of Formula (I) includes a compound and forms thereof, wherein
R₁ is selected from the group consisting of hydrogen, hydroxy, C₁₋₈alkoxy, amino-amino, phenyl-amino, morpholinyl-C₁₋₈alkoxy, piperidinyl-C₁₋₈alkyl-amino, morpholinyl-C₁₋₈alkyl-amino and morpholinyl-C₁₋₈alkyl-carbonyl-amino-amino,
wherein phenyl is optionally substituted with one substituent selected from the group consisting of halogen and C₁₋₈alkoxy.

Examples of a compound of Formula (I) include compounds selected from the group consisting of:

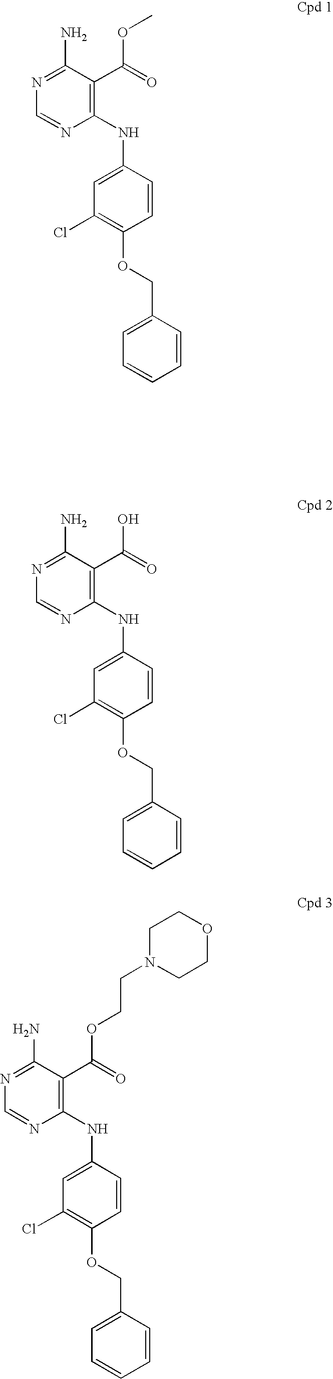

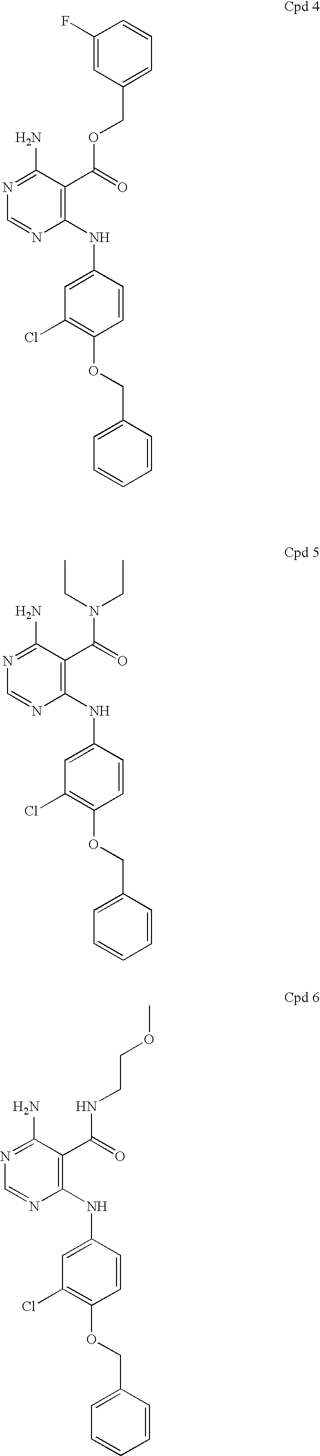

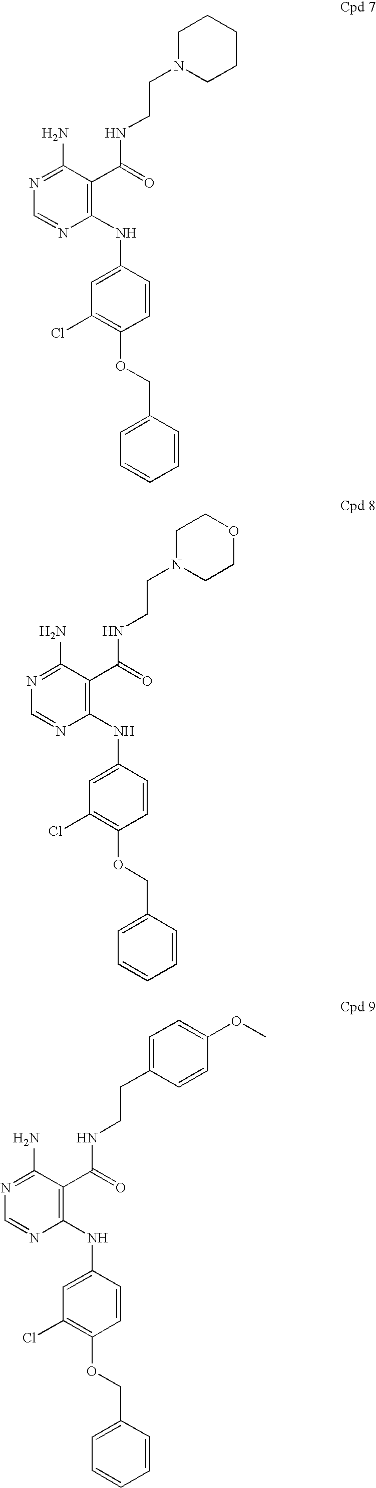

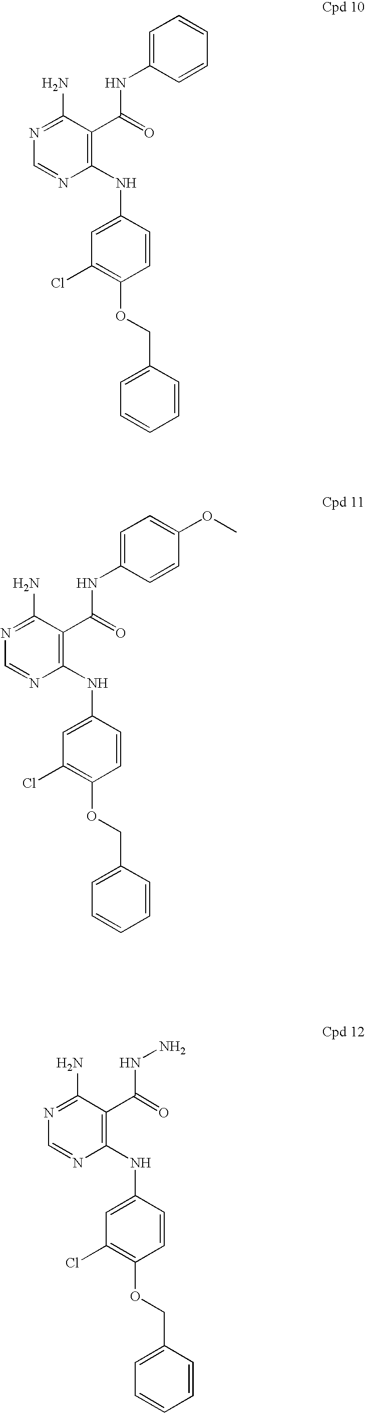

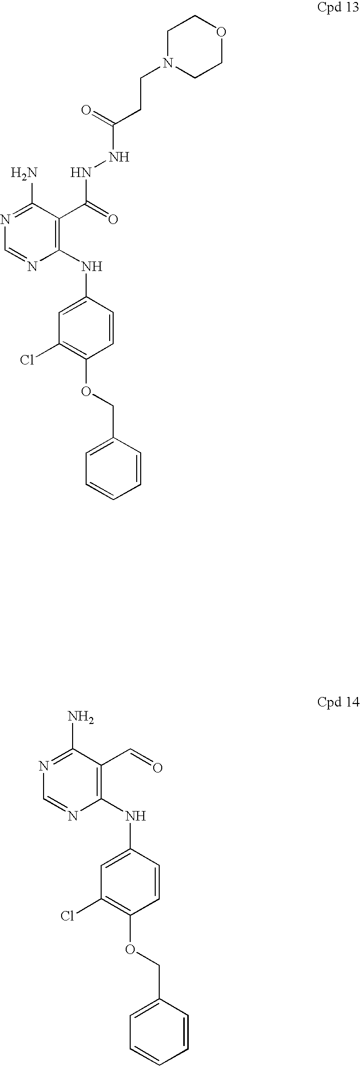

A representative compound of Formula (I) includes a compound selected from:

| Cpd | Name |
|---|---|
| 1 | 4-amino-6-(4-benzyloxy-3-chloro-phenylamino)-pyrimidine-5-carboxylic acid methyl |
| | ester, |
| 2 | 4-amino-6-(4-benzyloxy-3-chloro-phenylamino)-pyrimidine-5-carboxylic acid, |
| 3 | 4-amino-6-(4-benzyloxy-3-chloro-phenylamino)-pyrimidine-5-carboxylic |
| | acid 2-morpholin-4-yl-ethyl ester, |
| 4 | 4-amino-6-(4-benzyloxy-3-chloro-phenylamino)-pyrimidine-5-carboxylic |
| | acid 3-fluoro-benzyl ester, |
| 5 | 4-amino-6-(4-benzyloxy-3-chloro-phenylamino)-pyrimidine-5-carboxylic |
| | acid diethylamide, |
| 6 | 4-amino-6-(4-benzyloxy-3-chloro-phenylamino)-pyrimidine-5-carboxylic acid |
| | (2-methoxy-ethyl)-amide, |
| 7 | 4-amino-6-(4-benzyloxy-3-chloro-phenylamino)-pyrimidine-5-carboxylic |
| | acid (2-piperidin-1-yl-ethyl)-amide, |
| 8 | 4-amino-6-(4-benzyloxy-3-chloro-phenylamino)-pyrimidine-5-carboxylic |
| | acid (2-morpholin-4-yl-ethyl)-amide, |
| 9 | 4-amino-6-(4-benzyloxy-3-chloro-phenylamino)-pyrimidine-5-carboxylic |
| | acid [2-(4-methoxy-phenyl)-ethyl]-amide, |
| 10 | 4-amino-6-(4-benzyloxy-3-chloro-phenylamino)-pyrimidine-5-carboxylic acid |
| | phenylamide, |
| 11 | 4-amino-6-(4-benzyloxy-3-chloro-phenylamino)-pyrimidine-5-carboxylic |
| | acid (4-methoxy-phenyl)-amide, |
| 12 | 4-amino-6-(4-benzyloxy-3-chloro-phenylamino)-pyrimidine-5-carboxylic |
| | acid hydrazide, |
| 13 | 4-amino-6-(4-benzyloxy-3-chloro-phenylamino)-pyrimidine-5-carboxylic acid |
| | N′-(3-morpholin-4-yl-propionyl)-hydrazide, and |
| 14 | 4-amino-6-(4-benzyloxy-3-chloro-phenylamino)-pyrimidine-5-carboxaldehyde. |

A representative compound of Formula (I) includes a compound selected from:

| Cpd | Name |
|---|---|
| 1 | 4-amino-6-(4-benzyloxy-3-chloro-phenylamino)-pyrimidine-5- |
| | carboxylic acid methyl ester, |
| 2 | 4-amino-6-(4-benzyloxy-3-chloro-phenylamino)-pyrimidine-5- |
| | carboxylic acid, |
| 3 | 4-amino-6-(4-benzyloxy-3-chloro-phenylamino)-pyrimidine-5- |
| | carboxylic acid 2-morpholin-4-yl-ethyl ester, |
| 7 | 4-amino-6-(4-benzyloxy-3-chloro-phenylamino)-pyrimidine-5- |
| | carboxylic acid (2-piperidin-1-yl-ethyl)-amide, |
| 8 | 4-amino-6-(4-benzyloxy-3-chloro-phenylamino)-pyrimidine-5- |
| | carboxylic acid (2-morpholin-4-yl-ethyl)-amide, |
| 10 | 4-amino-6-(4-benzyloxy-3-chloro-phenylamino)-pyrimidine-5- |
| | carboxylic acid phenylamide, |
| 12 | 4-amino-6-(4-benzyloxy-3-chloro-phenylamino)-pyrimidine-5- |
| | carboxylic acid hydrazide, |
| 13 | 4-amino-6-(4-benzyloxy-3-chloro-phenylamino)-pyrimidine-5- |
| | carboxylic acid N′-(3-morpholin-4-yl-propionyl)-hydrazide, and |
| 14 | 4-amino-6-(4-benzyloxy-3-chloro-phenylamino)-pyrimidine-5- |
| | carboxaldehyde. |

A representative compound of Formula (I) includes a compound selected from:

| Cpd | Name |
|---|---|
| 1 | 4-amino-6-(4-benzyloxy-3-chloro-phenylamino)-pyrimidine-5- |
| | carboxylic acid methyl ester, |
| 2 | 4-amino-6-(4-benzyloxy-3-chloro-phenylamino)-pyrimidine-5- |
| | carboxylic acid, |
| 3 | 4-amino-6-(4-benzyloxy-3-chloro-phenylamino)-pyrimidine-5- |
| | carboxylic acid 2-morpholin-4-yl-ethyl ester, |
| 12 | 4-amino-6-(4-benzyloxy-3-chloro-phenylamino)-pyrimidine-5- |
| | carboxylic acid hydrazide, |
| 13 | 4-amino-6-(4-benzyloxy-3-chloro-phenylamino)-pyrimidine-5- |
| | carboxylic acid N′-(3-morpholin-4-yl-propionyl)-hydrazide, and |
| 14 | 4-amino-6-(4-benzyloxy-3-chloro-phenylamino)-pyrimidine-5- |
| | carboxaldehyde. |

A representative compound of Formula (I) includes a compound selected from:

| Cpd | Name |
|---|---|
| 1 | 4-amino-6-(4-benzyloxy-3-chloro-phenylamino)-pyrimidine-5- |
| | carboxylic acid methyl ester, |
| 2 | 4-amino-6-(4-benzyloxy-3-chloro-phenylamino)-pyrimidine-5- |
| | carboxylic acid, and |
| 14 | 4-amino-6-(4-benzyloxy-3-chloro-phenylamino)-pyrimidine-5- |
| | carboxaldehyde. |

A representative compound of Formula (I) includes a compound selected from:

| Cpd | Name |
|---|---|
| 2 | 4-amino-6-(4-benzyloxy-3-chloro-phenylamino)-pyrimidine-5-carboxylic acid, |
| 3 | 4-amino-6-(4-benzyloxy-3-chloro-phenylamino)-pyrimidine-5-carboxylic |
| | acid 2-morpholin-4-yl-ethyl ester, |
| 4 | 4-amino-6-(4-benzyloxy-3-chloro-phenylamino)-pyrimidine-5-carboxylic acid 3-fluoro- |
| | benzyl ester, |
| 5 | 4-amino-6-(4-benzyloxy-3-chloro-phenylamino)-pyrimidine-5-carboxylic acid |
| | diethylamide, |
| 6 | 4-amino-6-(4-benzyloxy-3-chloro-phenylamino)-pyrimidine-5-carboxylic |
| | acid (2-methoxy-ethyl)-amide, |
| 7 | 4-amino-6-(4-benzyloxy-3-chloro-phenylamino)-pyrimidine-5-carboxylic |
| | acid (2-piperidin-1-yl-ethyl)-amide, |
| 8 | 4-amino-6-(4-benzyloxy-3-chloro-phenylamino)-pyrimidine-5-carboxylic |
| | acid (2-morpholin-4-yl-ethyl)-amide, |
| 9 | 4-amino-6-(4-benzyloxy-3-chloro-phenylamino)-pyrimidine-5-carboxylic acid [2-(4- |
| | methoxy-phenyl)-ethyl]-amide, |
| 10 | 4-amino-6-(4-benzyloxy-3-chloro-phenylamino)-pyrimidine-5-carboxylic |
| | acid phenylamide, and |
| 11 | 4-amino-6-(4-benzyloxy-3-chloro-phenylamino)-pyrimidine-5-carboxylic acid (4- |
| | methoxy-phenyl)-amide. |

A representative compound of Formula (I) includes a compound selected from:

| Cpd | Name |
|---|---|
| 2 | 4-amino-6-(4-benzyloxy-3-chloro-phenylamino)-pyrimidine-5- |
| | carboxylic acid, |
| 3 | 4-amino-6-(4-benzyloxy-3-chloro-phenylamino)-pyrimidine-5- |
| | carboxylic acid 2-morpholin-4-yl-ethyl ester, |
| 7 | 4-amino-6-(4-benzyloxy-3-chloro-phenylamino)-pyrimidine-5- |
| | carboxylic acid (2-piperidin-1-yl-ethyl)-amide, |
| 8 | 4-amino-6-(4-benzyloxy-3-chloro-phenylamino)-pyrimidine-5- |
| | carboxylic acid (2-morpholin-4-yl-ethyl)-amide, and |
| 10 | 4-amino-6-(4-benzyloxy-3-chloro-phenylamino)-pyrimidine-5- |
| | carboxylic acid phenylamide. |

A representative compound of Formula (I) includes a compound selected from:

| Cpd | Name |
|---|---|
| 2 | 4-amino-6-(4-benzyloxy-3-chloro-phenylamino)-pyrimidine-5- |
| | carboxylic acid, and |
| 3 | 4-amino-6-(4-benzyloxy-3-chloro-phenylamino)-pyrimidine-5- |
| | carboxylic acid 2-morpholin-4-yl-ethyl ester. |

### Chemical Definitions & Nomenclature

Bond lines drawn into a ring system from a substituent variable indicate that the substituent may be attached to any of the substitutable ring atoms.

As used herein, the following terms are intended to have the following definitions. The definitions herein may specify that a chemical term has an indicated formula. The particular formula provided is not intended to limit the scope of the invention, but is provided as an illustration of the term. The scope of the per se definition of the term is intended to include the plurality of variations expected to be included by one of ordinary skill in the art. Chemical terms are to be read from right to left, wherein the right-most group is attached to the core molecule and the left-most group is the terminal group. The formula (s) illustrating a term are to be read from left to right, wherein the left-most group is attached to the core molecule, as indicated by the dash, and the right-most group is the terminal group.

The term “C₁₋₈alkyl” means a saturated aliphatic branched or straight-chain hydrocarbon radical or linking group having from 1 up to 8 carbon atoms in a linear or branched arrangement, wherein the radical is derived by the removal of one hydrogen atom from a carbon atom and the linking group is derived by the removal of one hydrogen atom from each of two carbon atoms in the chain. The term “C₁₋₈alkyl” also includes a “C₁₋₆alkyl” and “C₁₋₄alkyl” radical or linking group having from 1 up to 6 carbon atoms and 1 up to 4 carbon atoms respectively, such as methyl, ethyl, 1-propyl, 2-propyl, 1-butyl, 2-butyl, tert-butyl, 1-pentyl, 2-pentyl, 3-pentyl, 1-hexyl, 2-hexyl, 3-hexyl, 1-heptyl, 2-heptyl, 3-heptyl, 1-octyl, 2-octyl, 3-octyl and the like. Alkyl radicals may be attached to a core molecule via a terminal carbon atom or via a carbon atom within the chain. Similarly, substituent variables may be attached to an alkyl linking group when allowed by available valences.

The term “C₂₋₈alkenyl” means an alkyl radical or linking group having from 2 up to 8 carbon atoms in a linear or branched arrangement having at least one carbon-carbon double bond. The term “C₂₋₈ alkenyl” also includes a “C₂₋₄alkenyl” radical or linking group having from 2 up to 4 carbon atoms, such as ethenyl (also referred to as vinyl), iso-propenyl, allyl (also referred to as propenyl), propylidene and the like.

The term “C₁₋₈alkoxy” means an alkyl radical or linking group having from 1 up to 8 carbon atoms in a linear or branched arrangement, wherein the radical or linking group is attached through an oxygen linking atom, as in the formula: —O—C₁₋₈alkyl. The term “C₁₋₈alkoxy” also includes a “C₁₋₆alkoxy” and “C₁₋₄alkoxy” radical or linking group having from 1 up to 6 carbon atoms and from 1 up to 4 carbon atoms respectively, such as methoxy, ethoxy, propoxy, butoxy and the like. An alkoxy radical may be attached to a core molecule and farther substituted as a linking group where indicated.

The term “C₃₋₁₂cycloalkyl” means a saturated or partially unsaturated cyclic hydrocarbon ring system radical, The term “C₃₋₁₂cycloalkyl” also includes a C₃₋₈cycloalkyl, C₃₋₁₀cycloalkyl, C₅₋₆cycloalkyl, C₅₋₈cycloalkyl, C₅₋₁₂cycloalkyl, C₉₋₁₃cycloalkyl or benzofused-C₃₋₁₂cycloalkyl ring system radical and the like, such as cyclopropyl, cyclobutyl, cyclopentyl, cyclohexyl, cycloheptyl, cyclooctyl, 1H-indenyl, indanyl, 9H-fluorenyl, 1,2,3,4-tetrahydro-naphthalenyl, acenaphthenyl, adamantanyl and the like.

The term “benzofused-C₃₋₁₂cycloalkyl” means a C₃₋₁₂cycloalkyl ring system radical having a benzene ring fused on the ring system on adjacent carbons. Examples of benzofused-C₃₋₁₂cycloalkyl in compounds representative of the present invention include a benzofused-C₅₋₆cycloalkyl ring system radical and the like, such as 1H-indenyl, indanyl and the like.

The term “aryl” means an unsaturated aromatic hydrocarbon ring system radical. Aryl ring systems include phenyl, naphthalenyl, azulenyl, anthracenyl and the like. Examples of aryl in compounds representative of the present invention include phenyl or naphthalenyl.

The term “hetero”, when used as a prefix for a ring system, refers to the replacement of at least one carbon atom member in the ring system with a heteroatom selected from N, O, S, S(O), or SO₂. A hetero ring may have 1, 2, 3 or 4 carbon atom members replaced by a nitrogen atom. Alternatively, a ring may have 1, 2 or 3 nitrogen atom members and 1 oxygen or sulfur atom member. Alternatively, a ring may have 1 oxygen or sulfur atom member. Alternatively, up to two adjacent ring members may be heteroatoms, wherein one heteroatom is nitrogen and the other heteroatom is selected from N, S or O.

The term “heterocyclyl” means a saturated or partially unsaturated “hetero” ring system radical. Heterocyclyl ring systems include azetidinyl, 2H-pyrrole, 2-pyrrolinyl, 3-pyrrolinyl, pyrrolidinyl, 1,3-dioxolanyl, 2-imidazolinyl (also referred to as 4,5-dihydro-1H-imidazolyl), imidazolidinyl, 2-pyrazolinyl, pyrazolidinyl, tetrazolyl, tetrazolidinyl, piperidinyl, 1,4-dioxanyl, morpholinyl, 1,4-dithianyl, thiomorpholinyl, piperazinyl, azepanyl, hexahydro-1,4-diazepinyl, hexahydro-1,4-oxazepanyl, tetrahydro-furanyl, tetrahydro-thienyl, tetrahydro-pyranyl, tetrahydro-pyridazinyl and the like. The term “heterocyclyl” also includes a benzofused-heterocyclyl ring system radical and the like, such as indolinyl (also referred to as 2,3-dihydro-indolyl), benzo[1,3]dioxolyl, 2,3-dihydro-1,4-benzodioxinyl, 2,3-dihydro-benzofuranyl, 1,2-dihydro-phthalazinyl and the like.

The term “benzofused-heterocyclyl” means a heterocyclyl ring system radical having a benzene ring fused on the ring system on adjacent carbons. Examples of benzofused-heterocyclyl in compounds representative of the present invention include benzo[1,3]dioxolyl and 2,3-dihydro-indolyl.

The term “heteroaryl” means a monovalent, unsaturated aromatic “hetero” ring system radical. Heteroaryl ring systems include furyl, thienyl, pyrrolyl, oxazolyl, thiazolyl, imidazolyl, pyrazolyl, isoxazolyl, isothiazolyl, oxadiazolyl, triazolyl, thiadiazolyl, pyridinyl, pyridazinyl, pyrimidinyl, pyrazinyl and the like.

The term “heteroaryl” also includes a benzofused-heteroaryl ring system radical and the like, such as indolizinyl, indolyl, azaindolyl, isoindolyl, benzofuranyl, benzothienyl, indazolyl, azaindazolyl, benzoimidazolyl, benzothiazolyl, benzoxazolyl, benzoisoxazolyl, benzothiadiazolyl, benzotriazolyl, purinyl, 4H-quinolizinyl, quinolinyl, isoquinolinyl, cinnolinyl, phthalazinyl, quinazolinyl, quinoxalinyl, 1,8-naphthyridinyl, pteridinyl and the like.

The term “benzofused-heteroaryl” means a heteroaryl ring system radical having a benzene ring fused on the ring system on adjacent carbons. Examples of benzofused-heteroaryl in compounds representative of the present invention include indazolyl, indolyl, benzofuranyl and benzoimidazolyl.

The term “C₁₋₈acyl” means a radical of the formula: —C(O)H or —C(O)—C₁₋₈alkyl.

The term “C₁₋₈acyl-amino” means a radical of the formula: —NH—C(O)H or —NH—C(O)—C₁₋₈alkyl.

The term “C₁₋₈alkoxy-C₁₋₈alkyl” means a radical of the formula: —C₁₋₈alkyl-O—C₁₋₈alkyl.

The term “C₁₋₈alkoxy-C₁₋₈alkyl-amino” means a radical of the formula: —NH—C₁₋₈alkyl-O—C₁₋₈alkyl or —N(C₁₋₈alkyl-O—C₁₋₈alkyl)₂.

The term “C₁₋₈alkoxy-C₁₋₈alkyl-amino-C₁₋₈alkyl” means a radical of the formula: —C₁₋₈alkyl-NH—C₁₋₈alkyl-O—C₁₋₈alkyl or —C₁₋₈alkyl-N(C₁₋₈alkyl-O—C₁₋₈alkyl)₂.

The term “C₁₋₈alkoxy-carbonyl” means a radical of the formula: —C(O)—O—C₁₋₈alkyl.

The term “C₁₋₈alkoxy-imino-C₁₋₈alkyl” means a radical of the formula: —C₁₋₈alkyl=N(C₁₋₈alkoxy).

The term “C₁₋₈alkoxy-imino-(aryl)C₁₋₈alkyl” means a radical of the formula: —C₁₋₈alkyl(aryl)-N(C₁₋₈alkoxy); wherein the aryl and imino portion is substituted on the same or different C₁₋₈alkyl carbon atom.

The term “C₁₋₈alkyl-amino” means a radical of the formula: —NH—C₁₋₈alkyl or —N(C₁₋₈alkyl)₂.

The term “C₁₋₈alkyl-amino-C₁₋₈alkyl” means a radical of the formula: —C₁₋₈alkyl-NH—C₁₋₈alkyl or —C₁₋₈alkyl-N(C₁₋₈alkyl)₂.

The term “C₁₋₈alkyl-amino-C₁₋₈alkyl-amino-carbonyl” means a radical of the formula: —C(O)—NH—C₁₋₈alkyl-NH—C₁₋₈alkyl or —C(O)—NH—C₁₋₈alkyl-N(C₁₋₈alkyl)₂.

The term “C₁₋₈alkyl-carbonyl” means a radical of the formula: —C(O)—C-s alkyl.

The term “C₁₋₈alkyl-sulfonyl-C₁₋₈alkyl” means a radical of the formula: —C₁₋₈alkyl-SO₂—C₁₋₈alkyl.

The term “C₁₋₈alkyl-sulfonyloxy-C₁₋₈alkyl” means a radical of the formula: —C₁₋₈alkyl-O—SO₂—C₁₋₈alkyl.

The term “amino” means a radical of the formula: —NH₂.

The term “amino-amino” means a radical of the formula: —NH—NH₂.

The term “amino-C₁₋₈alkyl” means a radical of the formula: —C₁₋₈alkyl-NH₂.

The term “aryl-C₁₋₈alkyl” means a radical of the formula: —C₁₋₈alkyl-aryl.

The term “aryl-amido” means a radical of the formula: —NHC(O)-aryl.

The term “aryl-amino” means a radical of the formula: —NH-aryl.

The term “aryl-carbonyl-C₁₋₈alkyl” means a radical of the formula: —C₁₋₈alkyl-C(O)-aryl.

The term “aryl-C₁₋₈alkoxy” means a radical of the formula: —O—C₁₋₈alkyl-aryl.

The term “aryl-C₁₋₈alkyl-amino” means a radical of the formula: —NH—C₁₋₈alkyl-aryl or —N(C₁₋₈alkyl-aryl)₂.

The term “aryloxy” means a radical of the formula: —O-aryl.

The term “aryloxy-C₁₋₈alkyl” means a radical of the formula: —C₁₋₈alkyl-O-aryl.

The term “benzofused-heterocyclyl-C₁₋₈alkyl” means a radical of the formula: —C₁₋₈alkyl-(benzofused-heterocyclyl).

The term “carboxy” means a radical of the formula: —C(O)OH.

The term “cyano-C₁₋₈alkyl” means a radical of the formula: —C₁₋₈alkyl-C≡N.

The term “halogen” or “halo” means the group chloro, bromo, fluoro or iodo.

The term “halo-C₁₋₈alkoxy” means a radical of the formula: —C₁₋₈alkoxy(halo)₁₋₁₇, wherein one or more halogen atoms may be substituted on C₁₋₈alkoxy when allowed by available valences and includes monofluoromethoxy, difluoromethoxy, trifluoromethoxy, trifluoroethoxy and the like.

The term “halo-C₁₋₆alkoxy” means a radical of the formula: —C₁₋₆alkoxy(halo)₁₋₁₃, wherein one or more halogen atoms may be substituted on C₁₋₆alkoxy when allowed by available valences.

The term “halo-C₁₋₈alkyl” means a radical of the formula: —C₁₋₈alkyl(halo)₁₋₁₇, wherein one or more halogen atoms may be substituted on C₁₋₈alkyl when allowed by available valences and includes monofluoromethyl, difluoromethyl, trifluoromethyl, trifluoroethyl and the like.

The term “halo-C₁₋₆alkyl” means a radical of the formula: —C₁₋₆alkyl(halo)₁₋₁₃, wherein one or more halogen atoms may be substituted on C₁₋₆alkyl when allowed by available valences.

The term “heterocyclyl-C₁₋₈alkoxy” means a radical of the formula: —O—C₁₋₈alkyl-heterocyclyl.

The term “heterocyclyl-C₁₋₈alkyl” means a radical of the formula: —C₁₋₈alkyl-heterocyclyl.

The term “heterocyclyl-C₁₋₈alkyl-amino” means a radical of the formula: —NH—C₁₋₈alkyl-heterocyclyl.

The term “heterocyclyl-carbonyl-C₁₋₈alkyl” means a radical of the formula: —C₁₋₈alkyl-C(O)-heterocyclyl.

The term “heterocyclyl-C₁₋₈alkyl-carbonyl” means a radical of the formula: —(O)—C₁₋₈alkyl-heterocyclyl.

The term “heterocyclyl-C₁₋₈alkyl-carbonyl-amino” means a radical of the formula: —NH—(O)—C₁₋₈alkyl-heterocyclyl.

The term “heterocyclyl-C₁₋₈alkyl-carbonyl-amino-amino” means a radical of the formula: —NH—NH—(O)—C₁₋₈alkyl-heterocyclyl.

The term “heteroaryl-C₁₋₈alkoxy” means a radical of the formula: —O—C₁₋₈alkyl-heteroaryl.

The term “heteroaryl-C₁₋₈alkyl” means a radical of the formula: —C₁₋₈alkyl-heteroaryl.

The term “heteroaryl-amino-sulfonyl” means a radical of the formula: —SO₂—NH-heteroaryl.

The term “heteroaryloxy” means a radical of the formula: —O-heteroaryl.

The term “hydroxy-C₁₋₈alkoxy” means a radical wherein C₁₋₈alkoxy is substituted on an available carbon chain atom with one or more hydroxy radicals.

The term “hydroxy-C₁₋₈alkyl” means a radical wherein C₁₋₈alkyl is substituted on an available carbon chain atom with one or more hydroxy radicals.

The term “substituted phosphonic acid” means a radical of the formula: —P[(═O)(O—C₁₋₈alkyl)₂], —P[(═O)(OH)₂] or —P[(═O)(OH)(—O—C₁₋₈alkyl)].

The term “thio-C₁₋₈alkyl” means a radical of the formula: —S—C₁₋₈alkyl.

The term “substituted” means the independent replacement of one or more hydrogen atoms within a radical with that amount of substituents allowed by available valences.

The term “dependently selected” means that the structure variables are specified in an indicated combination.

In general, IUPAC nomenclature rules are used herein.

### Compound Forms

The term “form” means, in reference to compounds of the present invention, such may exist as, without limitation, a salt, stereoisomer, tautomer, crystalline, polymorph, amorphous, solvate, hydrate, ester, prodrug or metabolite form. The present invention encompasses all such compound forms and mixtures thereof.

The term “isolated form” means, in reference to compounds of the present invention, such may exist in an essentially pure state such as, without limitation, an enantiomer, a racemic mixture, a geometric isomer (such as a cis or trans stereoisomer), a mixture of geometric isomers, and the like. The present invention encompasses all such compound forms and mixtures thereof.

The compounds of the invention may be present in the form of pharmaceutically acceptable salts. For use in medicines, the “pharmaceutically acceptable salts” of the compounds of this invention refer to non-toxic acidic/anionic or basic/cationic salt forms.

Suitable salt forms include acid addition salts which may, for example, be formed by mixing a solution of the compound according to the invention with a solution of an acid such as acetic acid, adipic acid, benzoic acid, carbonic acid, citric acid, fumaric acid, glycolic acid, hydrochloric acid, maleic acid, malonic acid, phosphoric acid, saccharinic acid, succinic acid, sulphuric acid, tartaric acid, trifluoroacetic acid and the like.

Furthermore when the compounds of the present invention carry an acidic moiety, suitable salts thereof may include alkali metal salts, e.g. sodium or potassium salts; alkaline earth metal salts, e.g. calcium or magnesium salts; and salts formed with suitable organic ligands, e.g. quaternary ammonium salts.

Thus, representative salts include the following: acetate, adipate, benzenesulfonate, benzoate, bicarbonate, bisulfate, bitartrate, borate, bromide, calcium, camsylate (or camphorsulphonate), carbonate, chloride, clavulanate, citrate, dihydrochloride, edetate, fumarate, gluconate, glutamate, glyconate, hydrabamine, hydrobromine, hydrochloride, iodide, isothionate, lactate, malate, maleate, malonate, mandelate, mesylate, nitrate, oleate, pamoate, palmitate, phosphate/diphosphate, saccharinate, salicylate, stearate, sulfate, succinate, tartrate, tosylate, trichloroacetate, trifluoroacetate and the like.

Examples of salt forms of compounds representative of the present invention include the monohydrochloride salt.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, 3^{rd} Edition, John Wiley & Sons, 1999. The protecting groups may be removed at a convenient subsequent stage using methods known in the art. The scope of the present invention encompasses all such protected compound forms and mixtures thereof.

The invention includes compounds of various isomers and mixtures thereof. The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (optical isomers).

The term “optical isomer” means isomers of identical constitution that differ only in the spatial arrangement of their groups. Optical isomers rotate the plane of polarized light in different directions. The term “optical activity” means the degree to which an optical isomer rotates the plane of polarized light.

The term “racemate” or “racemic mixture” means an equimolar mixture of two enantiomeric species, wherein each of isolated specie rotates the plane of polarized light in the opposite direction such that the mixture is devoid of optical activity.

The term “enantiomer” means an isomer having a nonsuperimposabie mirror image. The term “diastereomer” means stereoisomers that are not enantiomers.

The term “chiral” means a molecule which, in a given configuration, cannot be superimposed on its mirror image. This is in contrast to achiral molecules which can be superimposed on their mirror images.

The two distinct mirror image versions of the chiral molecule are also known as levo (left-handed), abbreviated L, or dextro (right handed), abbreviated 1, depending on which way they rotate polarized light. The symbols “R” and “S” represent the configuration of groups around a stereogenic carbon atom(s).

An example of an isolated form of an achiral mixture includes a dextrorotatory enantiomer, wherein the mixture is substantially free of the levorotatory isomer. In this context substantially free means the levorotatory isomer may, in a range, comprise less than 25% of the mixture, less than 10%, less than 5%, less than 2% or less than 1% of the mixture according to the formula:

$$\% levorotatory = \frac{( mass levorotatory )}{( mass dextrorotatory ) + ( mass levorotatory )} \times 100$$

Similarly, an example of an isolated form of an achiral mixture includes a levorotatory enantiomer, wherein the mixture is substantially free of the dextrorotatory isomer. In this context, substantially free means the dextrorotatory isomer may, in a range, comprise less than 25% of the mixture, less than 10%, less than 5%, less than 2% or less than 1% of the mixture according to the formula:

$$\% dextrorotatory = \frac{( mass dextrorotatory )}{( mass dextrorotatory ) + ( mass levorotatory )} \times 100$$

The term “geometric isomer” means isomers that differ in the orientation of substituent atoms in relationship to a carbon-carbon double bond, to a cycloalkyl ring, or to a bridged bicyclic system. Substituent atoms (other than hydrogen) on each side of a carbon-carbon double bond may be in an E or Z configuration. In the “E” configuration, the substituents are on opposite sides in relationship to the carbon-carbon double bond. In the “Z” configuration, the substituents are oriented on the same side in relationship to the carbon-carbon double bond.

Substituent atoms (other than hydrogen) attached to a ring system may be in a cis or trans configuration. In the “cis” configuration, the substituents are on the same side in relationship to the plane of the ring; in the “trans” configuration, the substituents are on opposite sides in relationship to the plane of the ring. Compounds having a mixture of “cis” and “trans” species are designated “cis/trans”.

The isomeric descriptors (“R,” “S,” “E,” and “Z”) indicate atom configurations and are intended to be used as defined in the literature.

The compounds of the invention may be prepared as individual isomers by either isomer-specific synthesis or resolved from an isomeric mixture. Conventional resolution techniques include combining the free base (or free acid) of each isomer of an isomeric pair using an optically active acid (or base) to form an optically active salt (followed by fractional crystallization and regeneration of the free base), forming an ester or amide of each of the isomers of an isomeric pair by reaction with an appropriate chiral auxiliary (followed by fractional crystallization or chromatographic separation and removal of the chiral auxiliary), or separating an isomeric mixture of either an intermediate or a final product using various well known chromatographic methods.

Furthermore, compounds of the present invention may have one or more polymorph or amorphous crystalline forms and, as such, are intended to be included in the scope of the invention. In addition, some of the compounds may form solvates with water (i.e., hydrates) or common organic solvents (e.g., organic esters such as ethanolate and the like) and, as such, are also intended to be encompassed within the scope of this invention.

### Methods of Use

The compounds of Formula (I) are inhibitors of a protein kinase such as EGFR, HER-1, HER-2 and the like, having an IC₅₀ (50% inhibition concentration) or an EC₅₀ (50% effective concentration) in a range of about 50 μM or less, of about 25 μM or less, of about 15 μM or less, of about 10 μM or less, of about 5 μM or less, of about 1 μM or less, of about 0.5 μM or less, of about 0.25 μM or less or of about 0.1 μM or less.

The present invention includes a compound of Formula (I) and forms thereof as a protein kinase inhibitor, wherein the protein kinase is selected from EGFR, HER-1 or HER-2.

The present invention includes a prodrug form of a compound of Formula (I) and forms thereof as a protein kinase inhibitor.

The present invention includes a metabolite form of a compound of Formula (I) and forms thereof as a protein kinase inhibitor.

The present invention includes an isolated form of a compound of Formula (I) and forms thereof as a protein kinase inhibitor.

The present invention includes a compound of Formula (I) or a form thereof, wherein the compound is labeled with a ligand for use as a marker, and wherein the ligand is a radioligand selected from deuterium, tritium and the like.

The present invention includes use of a compound of Formula (I) and forms thereof as an inhibitor of a protein kinase such as EGFR, HER-1, HER-2 and the like comprising contacting the protein kinase domain or receptor with the compound.

The present invention includes the use of a compound of Formula (I) and forms thereof as a pharmaceutical composition, medicine or medicament for treating, preventing or ameliorating a kinase mediated disease, disorder or condition.

The present invention includes the use of a compound of Formula (I) and forms thereof as a medicament.

The present invention includes the use of a prodrug of a compound of Formula (I) and forms thereof as a pharmaceutical composition, medicine or medicament for treating, preventing or ameliorating a kinase mediated disease, disorder or condition.

The present invention includes the use of a prodrug of a compound of Formula (I) and forms thereof as a medicament.

The present invention is directed to a method for treating, preventing or ameliorating a chronic or acute protein kinase mediated disease, disorder or condition in a subject in need thereof comprising administering to the subject an effective amount of a compound of Formula (I) and forms thereof.

The method of the present invention further comprises administering to the subject an effective amount of a prodrug of a compound of Formula (I) and forms thereof.

The method of the present invention further comprises treating, preventing or ameliorating a chronic or acute EGFR, HER-1 or HER-2 mediated disease, disorder or condition.

The method of the present invention wherein the disease, disorder or condition is associated with increased or unregulated protein kinase activity, expression or signaling and the like in the subject.

The method of the present invention further comprises administering to the subject an effective amount of a compound of Formula (I) as a pharmaceutical composition, medicine or medicament thereof.

The method of the present invention wherein the disease, disorder or condition is an EGFR kinase mediated head or brain cancer in the subject, and wherein the compound penetrates the blood brain barrier.

The method of the present invention further comprises treating or ameliorating nerve damage and promoting axon regeneration subsequent to a brain or spinal cord injury in the subject, wherein the compound is an EGFR inhibitor.

The method of the present invention further comprises treating, preventing or ameliorating viral infection by an ECFR kinase mediated cytomegalovirus in the subject.

The term “chronic or acute protein kinase mediated disease, disorder or condition” as used herein, includes, and is not limited to diseases, disorders or conditions associated with unregulated kinase activity and conditions that accompany such activity.

The term “unregulated protein kinase activity, expression or signaling” refers to 1) increased or unregulated kinase expression or signaling, 2) increased kinase expression leading to unregulated cell proliferation, 3) increased kinase signaling leading to unregulated cell proliferation, or 4) mutations leading to constitutive kinase activation. The existence of unregulated kinase activity may be determined by procedures well known in the art.

The term “unregulated cell proliferation” refers to cell proliferation of one or more subset of cells in a multicellular organism resulting in harm (such as discomfort or decreased life expectancy) to the multicellular organism.

Tumor cells which result from unregulated cell proliferation use many mechanisms to enhance their survival and spread and often have high rates of proliferation because growth control signals that keep normal cells in check are defective. Many tumor cells secrete autocrine growth factors that increase proliferation rates or they induce other cells to secrete growth factors that they utilize.

Tumor cells grow and spread by dislodging from a primary tumor site, using proteases to digest the extracellular matrix, spreading in response to migration cues, allowing them to migrate to certain tissues preferentially where overexpressed adhesion molecules allow attachment and growth at the new site. The totality of these and other biological processes are responsible for the lethal effects of a tumor. A kinase inhibitor may affect one or more aspects of tumor survival mechanisms and thus be therapeutically useful. Alternatively, a kinase inhibitor may not affect one particular tumor survival mechanism but may still be therapeutically useful by affecting tumor survival by an unknown or as yet unelucidated mechanism of action.

The foregoing methods contemplate that a compound of Formula (I) or a form thereof is useful for treating, preventing or ameliorating diseases, disorders or conditions such as, without limitation, osteoarthritis, rheumatoid arthritis, synovial pannus invasion in arthritis, multiple sclerosis, myasthenia gravis, diabetes mellitus, diabetic angiopathy, diabetic retinopathy, retinal vessel proliferation, inflammatory bowel disease, Crohn's disease, ulcerative colitis, bone diseases, transplant or bone marrow transplant rejection, lupus, chronic pancreatitis, cachexia, septic shock, fibroproliferative and differentiative skin diseases or disorders, central nervous system diseases, neurodegenerative diseases, disorders or conditions related to nerve damage and axon degeneration subsequent to a brain or spinal cord injury, acute or chronic cancer, ocular diseases, viral infections, heart disease, lung or pulmonary diseases or kidney or renal diseases.

Certain diseases, disorders or conditions further include, without limitation, acute or chronic cancer selected from bladder cancer, brain, head or neck cancer, breast cancer, colorectal cancer, endometrial cancer, epidermoid cancer, esophageal cancer, gastric cancer, glioma cancer, lung cancer, ovarian cancer, pancreatic cancer, prostate cancer, renal cell cancer, Kaposi's sarcoma, leukemia, lymphoma or papillocarcinoma; and, cancer-associated pathologies selected from abnormal cell proliferation, unregulated cell proliferation, tumor growth, tumor angiopathy, tumor angiogenesis, tumor vascularization or metastatic cancer cell invasion and migration.

Certain diseases, disorders or conditions further include, without limitation, fibroproliferative and differentiative skin diseases or disorders selected from papilloma formation, psoriasis, dermatitis, eczema, seborrhea or chemotherapy-induced alopecia; central nervous system diseases selected from Alzheimer's disease, Parkinson's disease or depression; ocular diseases selected from macular degeneration, diseases of the cornea or glaucoma; viral infections selected from mycotic infection, autoimmune disease or cytomegalovirus; heart disease selected from atherosclerosis, neointima formation or transplantation-induced vasculopathies such as arterial restenosis; lung or pulmonary diseases selected from allergic-asthma, lung fibrosis, pulmonary fibrosis or chronic obstructive pulmonary disorder; and, kidney or renal diseases selected from acute, subacute or chronic forms of glomerulonephritis or membranoproliferative glomerulonephritis, glomerulosclerosis, congenital multicystic renal dysplasia or kidney fibrosis.

Certain HER1 kinase mediated cancer includes, without limitation, bladder cancer, brain, head or neck cancer, breast cancer, cervical cancer, colorectal cancer, gastric cancer, glioma cancer, endometrial cancer, esophageal cancer, lung cancer, ovarian cancer, pancreatic cancer or renal cell cancer.

Certain HERS kinase mediated cancer includes, without limitation, bladder cancer, brain, head or neck cancer, breast cancer, colorectal cancer, gastric cancer, endometrial cancer, esophageal cancer, lung cancer, ovarian cancer, prostate cancer or renal cell cancer.

The term “administering,” with respect to the methods of the present invention, refers to a means for treating, ameliorating or preventing a disease, disorder or syndrome as described herein with a compound of Formula (I) or a form thereof which would obviously be included within the scope of the invention albeit not specifically disclosed for certain of said compounds.

Such methods include therapeutically or prophylactically administering an effective amount of compound of Formula (I) or a form thereof at different times during the course of a therapy or concurrently in a combination form. Such methods further include administering an effective amount of said compound with one or more agents at different times during the course of a therapy or concurrently in a combination form.

The term “prodrug” means a compound of Formula (I) or a form thereof that is converted in vivo into a functional derivative form that may contribute to therapeutic biological activity, wherein the converted form may be: 1) a relatively active form; 2) a relatively inactive form; 3) a relatively less active form; or, 4) any form which results, directly or indirectly, from such in vivo conversions.

Prodrugs are useful when said compound may be either too toxic to administer systemically, absorbed poorly by the digestive tract or broken down by the body before it reaches its target. Conventional procedures for the selection and preparation of suitable prodrug derivatives are described in, for example, “Design of Prodrugs”, ed. H. Bundgaard, Elsevier, 1985.

The term “metabolite” means a prodrug form of a compound of Formula (I) or a form thereof converted by in vivo metabolism or a metabolic process to a relatively less active functional derivative of said compound.

The term “subject” as used herein, refers to a patient, such as an animal, a mammal or a human, who has been the object of treatment, observation or experiment and is at risk of (or susceptible to) developing a disease or disorder or having a disease or disorder related to unregulated kinase activity.

The term “effective amount” refers to that amount of a compound of Formula (I) or a form, pharmaceutical composition, medicine or medicament thereof that elicits the biological or medicinal response (such as inhibiting activation of unregulated kinase activity) in a tissue system, animal or human, that is being sought by a researcher, veterinarian, medical doctor, or other clinician, which includes alleviation of the symptoms of the disease or disorder being treated.

The effective amount of said compound is from about 0.001 mg/kg/day to about 300 mg/kg/day.

The term “pharmaceutical composition” refers to a product containing a compound of Formula (I) or a form thereof, such as a product comprising the specified ingredients in the specified amounts, as well as any product which results, directly or indirectly, from such combinations of the specified ingredients in the specified amounts.

The term “medicament” or “medicine” refers to a product containing a compound of Formula (I) or a form thereof. The present invention includes use of such a medicament for treating, preventing or ameliorating a chronic or acute kinase mediated disease, disorder or condition.

The term “pharmaceutically acceptable” refers to molecular entities and compositions that are of sufficient purity and quality for use in the formulation of a pharmaceutical composition, medicine or medicament of the present invention and that, when appropriately administered to an animal or a human, do not produce an adverse, allergic or other untoward reaction. Since both human use (clinical and over-the-counter) and veterinary use are equally included within the scope of the present invention, a pharmaceutically acceptable formulation would include a pharmaceutical composition, medicine or medicament for either human or veterinary use.

The term “combination form” refers to the use of a combination product comprising a compound of Formula (I) or a form, pharmaceutical composition, medicine or medicament thereof and at least one therapeutic agent for treating, preventing or ameliorating a chronic or acute protein kinase mediated disease, disorder or condition.

Advantageously, the effective amount of a combination product for treating, preventing or ameliorating a chronic or acute protein kinase mediated disease, disorder or condition may be a reduced amount of either or both the compound or therapeutic agent compared to the effective amount of the compound or therapeutic agent otherwise recommended for treating, preventing or ameliorating the disease, disorder or condition. Therefore, it is contemplated that the compound is administered to the subject before, during or after the time the agent is administered.

The term “therapeutic agent” refers to chemotherapeutic agents used to treat a kinase mediated cancer or antiviral agents used to treat cytomegalovirus. Chemotherapeutic agents include and are not limited to anti-angiogenic agents, anti-tumor agents, cytotoxic agents, inhibitors of cell proliferation, radiation therapy and the like or a combination thereof.

The term “treating, preventing or ameliorating” refers, without limitation, to facilitating the eradication of, inhibiting the progression of or promoting stasis of a chronic or acute kinase mediated disease, disorder or condition.

The term “radiation therapy” refers to a therapy that comprises exposing the subject in need thereof to radiation. The present invention includes a method for administering a compound of Formula (I) or a form, pharmaceutical composition, medicine or medicament thereof in combination with radiation therapy. Procedures for administering such therapy are known to those skilled in the art. The appropriate scheme of radiation therapy will be similar to those already employed in clinical therapies wherein the radiation therapy is used alone or in combination with other chemotherapeutic agents.

The present invention includes a pharmaceutical composition comprising an admixture of a compound of Formula (I) or a form thereof and one or more pharmaceutically acceptable excipients.

The present invention includes a process for making a pharmaceutical composition, medicine or medicament comprising mixing a compound of Formula (I) or a form thereof and an optional pharmaceutically acceptable carrier. The present invention includes a pharmaceutical composition, medicine or medicament resulting from the process of mixing a compound of Formula (I) or a form thereof and an optional pharmaceutically acceptable carrier. Contemplated processes include both conventional and unconventional pharmaceutical techniques.

Said pharmaceutical composition, medicine or medicament may take a wide variety of forms to effectuate mode of administration, wherein the mode includes, and is not limited to, intravenous (both bolus and infusion), oral, nasal, transdermal, topical with or without occlusion, and via injection intraperitoneally, subcutaneously, intramuscularly, intratumorally, intracerebrally or intracranially. The composition, medicine or medicament may be in a dosage unit such as a tablet, pill, capsule, powder, granule, sterile parenteral solution or suspension, metered aerosol or liquid spray, drop, ampoule, auto-injector device or suppository for such administration modes.

Pharmaceutical compositions, medicines or medicaments suitable for oral administration include solid forms such as pills, tablets, caplets, capsules (each including immediate release, timed release and sustained release formulations), granules and powders; and, liquid forms such as solutions, syrups, elixirs, emulsions and suspensions. Forms useful for parenteral administration include sterile solutions, emulsions and suspensions. Alternatively, the pharmaceutical composition, medicine or medicament may be presented in a form suitable for once-weekly or once-monthly administration; for example, an insoluble salt of the active compound, such as the decanoate salt, may be adapted to provide a depot preparation for intramuscular injection.

The dosage form (tablet, capsule, powder, injection, suppository, teaspoonful and the like) containing the pharmaceutical composition, medicine or medicament contains an effective amount of the active ingredient necessary to be therapeutically or prophylactically effective as described above. The pharmaceutical composition, medicine or medicament may contain from about 0.001 mg to about 5000 mg (preferably, from about 0.001 to about 500 mg) of a compound of Formula (I) or a form thereof and may be constituted into any form suitable for the mode of administration selected for a subject in need.

An example of a contemplated effective amount for a pharmaceutical composition, medicine or medicament of the present invention may range from about 0.001 mg to about 300 mg/kg of body weight per day. In another example, the range is from about 0.003 to about 100 mg/kg of body weight per day. In another example, the range is from about 0.005 to about 15 mg/kg of body weight per day. The pharmaceutical composition, medicine or medicament may be administered according to a dosage regimen of from about 1 to about 5 times per day.

For oral administration, the pharmaceutical composition, medicine or medicament is preferably in the form of a tablet containing, e.g., 0.01, 0.05, 0.1, 0.5, 1.0, 2.5, 5.0, 10.0, 15.0, 25.0, 50.0, 100, 150, 200, 250 and 500 milligrams of a compound of Formula (I) or a form thereof for the symptomatic adjustment of the dosage to the patient to be treated. Optimal dosages will vary depending on factors associated with the particular patient being treated (e.g., age, weight, diet and time of administration), the severity of the condition being treated, the particular compound being used, the mode of administration and the strength of the preparation. The use of either daily administration or post-periodic dosing may be employed.

### Synthetic Methods

Representative compounds of the present invention can be synthesized in accordance with the general synthetic schemes described below and are illustrated more particularly in the specific synthetic examples that follow. The general schemes and specific examples are offered by way of illustration; the invention should not be construed as being limited by the chemical reactions and conditions expressed.

Except where indicated, starting materials and intermediates used in the schemes and examples are prepared by known methodologies well within the ordinary skill of persons versed in the art. No attempt has been made to optimize the yields obtained in any of the example reactions. One skilled in the art would also know how to increase such yields through routine variations in materials, solvents, reagents, reaction conditions and the like.

Compounds of the present invention can also be useful as intermediates for conversion to other compounds representative of the present invention via functional group transformations.

DSC analysis was conducted on a TA Instruments Q100. The calibration standard was indium. A sample (approximately 2 mg) was placed into a tared TA DSC pan and weight was recorded. Crimped pans were used for analysis and the samples were heated under nitrogen (50 cc/min) at a rate of 10° C./min, up to a final temperature of 250° C. The data were processed using a thermal analyzer (Universal Analyzer 2000, TA Instruments).

The terms used in describing the invention are commonly used and known to those skilled in the art. When used herein, the following abbreviations have the indicated meanings:

| Abbreviation | Meaning |
|---|---|
| Cpd | compound |
| DCM | dichloromethane |
| DMF | N,N-dimethylformamide |
| DMSO | dimethyl sulfoxide |
| EtOAc | ethyl acetate |
| HATU | O-(7-azabenzotriazol-1-yl)-N,N,N′,N′- |
| | tetramethyluronium hexafluorophosphate |
| hr(s)/min(s) | hour(s)/min(s) |
| LCMS Rt | High Pressure Liquid Chromatography Mass |
| | Spectrum Retention Time |
| RT/rt/r.t. | room temperature |
| TEA or Et₃N | triethylamine |

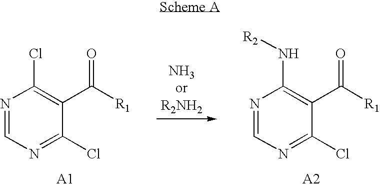

A solution of Compound A1 (wherein R₁ is hydrogen) in a suitable solvent such as toluene and the like is reacted with a volume of NH₃ gas or a solution of R₂NH₂ under suitable conditions to provide a Compound A2.

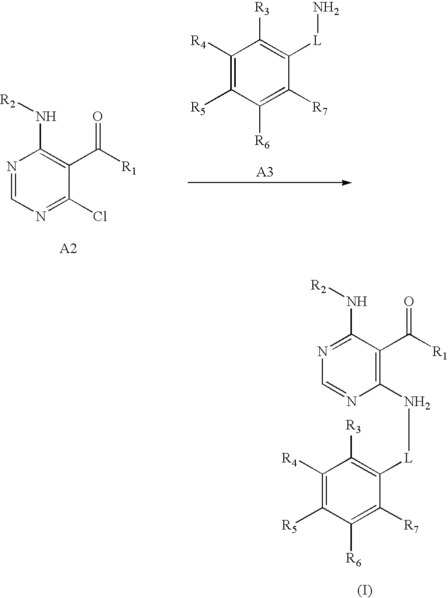

A mixture of Compound A2 (wherein R₁ is hydrogen), in a suitable solvent such as DMSO and the like, and a reagent (such as triethylamine and the like) is reacted with a Compound A3 to provide a compound of Formula (I), wherein R₁ is hydrogen.

An example of the invention includes converting a compound of Formula (I) (wherein R₁ is hydrogen) into additional examples of compounds of Formula (I) using suitable techniques and reagents known to those skilled in the art.

An example of the invention includes reacting a compound of Formula (I) (wherein R₁ is hydrogen) in a suitable solvent such as THF with a C₁₋₈alkyl-OH compound in a reagent mixture (such as a mixture of MnO₂, NaCN and AcOH) to provide a compound of Formula (I), wherein R₁ is C₁₋₈alkoxy.

Another example of the invention includes reacting a compound of Formula (I) (wherein R₁ is C₁₋₈alkoxy) in a suitable solvent, such as a mixture of water, THF and MeOH, with a metal hydroxide (such as a LiOH, NaOH and KOH) to provide a compound of Formula (I) wherein R₁ is hydroxy.

Another example of the invention includes reacting a compound of Formula (I) (wherein R₁ is hydroxy) with a compound selected from the group consisting of C₁₋₈alkyl-OH, hydroxy-C₁₋₈alkyl-O, aryl-C₁₋₈alkyl-OH, and heterocyclyl-C₁₋₈alkyl-OH in a reagent mixture (such as HATU and EtN(i-Pr)₂) in a solvent (such as in THF or DMF) to provide a compound of Formula (I), wherein R₁ is selected from the group consisting of C₁₋₈alkoxy, hydroxy-C₁₋₈alkoxy, aryl-C₁₋₈alkoxy and heterocyclyl-C₁₋₈alkoxy.

Another example of the invention includes reacting a compound of Formula (I) (wherein R₁ is hydroxy) with a substituted amine compound and a reagent mixture (such as HATU and EtN(i-Pr)₂) in a solvent (such as in THF or DMF) to provide a compound of Formula (I), wherein R₁ is selected from the group consisting of amino, C₁₋₈alkyl-amino, C₁₋₈alkoxy-C₁₋₈alkyl-amino, amino-amino, C₁₋₈alkyl-amino-amino, C₁₋₈alkyl-carbonyl-amino-amino, aryl-amino, aryl-C₁₋₈alkyl-amino, heterocyclyl-C₁₋₈alkyl-amino, heterocyclyl-C₁₋₈alkyl-carbonyl-amino, heterocyclyl-C₁₋₈alkyl-carbonyl-amino-amino, heterocyclyl, and benzofused-heterocyclyl.

An example of the invention includes reacting a suspension of a compound of Formula (I (wherein R₁ is C₁₋₈alkoxy) in a solvent such as 10% EtOH with a substituted hydrazine compound to provide a compound of Formula (I), wherein R₁ is selected from the group consisting of amino-amino and C₁₋₈alkyl-amino-amino.

An example of the invention includes reacting a compound of Formula (I) (wherein R₁ is selected from the group consisting of amino-amino and C₁₋₈alkyl-amino-amino) is reacted with a substituted carboxylic acid compound in a reagent mixture (such as EDCI in DMF and the like) to provide a compound of Formula (I), wherein R₁ is selected from the group consisting of C₁₋₈alkyl-carbonyl-amino-amino and heterocyclyl-C₁₋₈alkyl-carbonyl-amino-amino.

### EXAMPLE 1

### 4-amino-6-(4-benzyloxy-3-chloro-phenylamino)-pyrimidine-5-carboxaldehyde (Cpd 14)

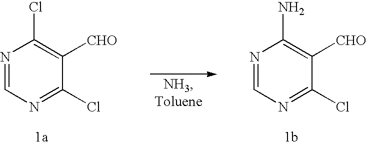

NH₃ (g) was blown through a solution of 4,6-dichloro-pyrimidine-5-carbaldehyde Compound 1a (50 g, 282.5 mmol) in toluene (565 mL, 0.5M) for 3 mins using a 12C glass frit, then the mixture was warmed at 60° C. with stirring for 30 min. NH₃ (g) was blown through the reaction mixture a second time for 3 min and the reaction was heated for 30 mins. NH₃ (g) was blown through the reaction mixture a third time for 3 min and the reaction was heated for a final 20 mins. The reaction mixture was diluted with H₂O (1 L), and extracted with EtOAc (1×750 mL, 3×500 mL). The organic extracts were washed with brine (4×) and dried (Na₂SO₄), then concentrated to afford 4-amino-6-chloro-pyrimidine-5-carbaldehyde Compound 1b (38.5 g, 87%) as a yellow solid. ¹H NMR (300 MHz, DMSO-d6) δ 10.27 (s, 1H), 8.74 (br S, 1H), 8.58 (br s, 1H), 8.42 (s, 1H).

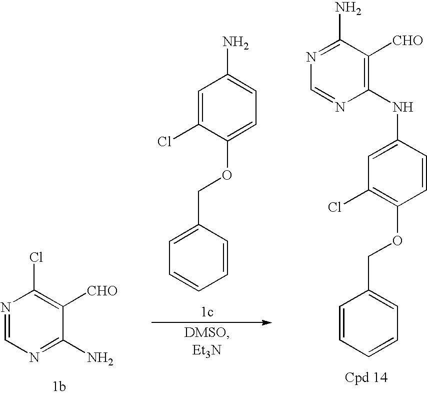

4-benzyloxy-3-chloro-phenylamine Compound 1c (8.9 g, 38 mmol) was added to a solution of Compound 1b (6.0 g, 38 mmol) and Et₃N (10.6 mL, 76 mmol) in DMSO (38 mL, 1M). The mixture was warmed at 100° C. for 3 hrs. The reaction mixture was cooled, then diluted with H₂O and extracted with EtOAc (3×). The organic extract was washed with H₂O (4×), concentrated onto SiO₂ (30 g) and purified via column chromatography (Horizon, 65+, 60 to 100% EtOAc/hexanes) to afford 4-amino-6-(4-benzyloxy-3-chloro-phenylamino)-pyrimidine-5-carbaldehyde Compound 14 (8.29 g, 61%) as a yellow solid. H E NMR (400 Mhz, CD₃OD, warm) δ 10.15 (s, 1H), 8.05 (s, 1H), 7.77 (d, J=2.4 Hz, 1H), 7.48 (d, J=76 Hz, 2H), 7.38 (m, 4H), 7.11 (d, J=8.8 Hz, 1H), 5.18, (s, 2H).

### 4-amino-6-(4-benzyloxy-3-chloro-phenylamino)-pyrimidine-5-carboxylic Acid Methyl Ester (Cpd 1)

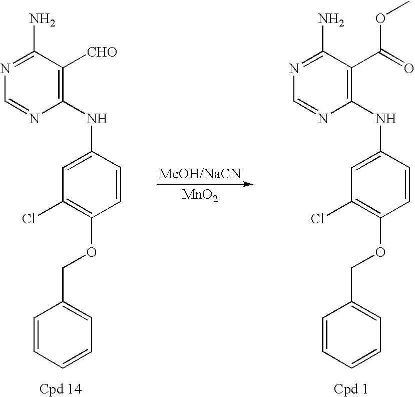

4-Amino-6-(4-benzyloxy-3-chloro-phenylamino)-pyrimidine-5-carbaldehyde Compound 14 (5.0 g, 14.1 mmol) in THF/MeOH (1/1, 125 mL) was treated with MnO₂ (7.4 g, 84.5 mmol), NaCN (2.1 g, 42.3 mmol), AcOH (2.4 mL, 42.3 mmol) and the reaction mixture was warmed at reflux for 24 h. The mixture was cooled to rt and filtered through Celite. The filtrate was diluted with H₂O (200 mL) and then extracted with a (1/1) mixture of EtOAc/THF (3×100 mL). The resulting organic extracts were dried (Na₂SO₄), and concentrated to afford 4-amino-6-(4-benzyloxy-3-chloro-phenylamino)-pyrimidine-5-carboxylic acid methyl ester Compound 1 (4.33 g, 80%) as a yellow solid. ¹H NMR (300 MHz, DMSO-d6) δ 10.26 (s, 1H), 8.05 (s, 14H), 7.86 (d, J=2.4 Hz, 1H), 7.64 (br s, 2H), 7.51-7.35 (m, 7H), 7.20 (d, J=8.7 Hz, 1H), 5.22 (s, 2H), 3.89 (s, 3H).

### EXAMPLE 3

### 4-amino-6-(4-benzyloxy-3-chloro-phenylamino)-pyrimidine-5-carboxylic Acid (Cpd 2)

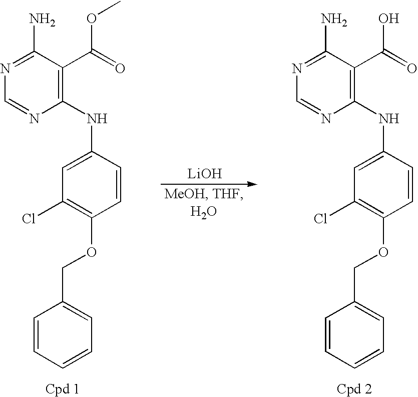

Lithium hydroxide (0.127 g, 6.3 mmol) was added to a suspension of 4-amino-6-(4-benzyl oxy-3-chloro-phenylamino)-pyrimidine-5-carboxylic acid methyl ester Compound 1 (0.924 g, 2.40 mmol) in 25 mL of a 3:1:1 mixture of THF:MeOH:water and the mixture was refluxed for 1.5 h. After cooling, 1 N aqueous HCl was added to bring the pH of the solution to approximately 4. The mixture was extracted with ethyl acetate/THF, resulting in the formation of a solid precipitate and two liquid layers, The aqueous liquid layer was removed and the organic liquid layer and solids were filtered, yielding 0.573 g (64%) of 4-amino-6-(4-benzyloxy-3-chloro-phenylamino)-pyrimidine-5-carboxylic acid Compound 2 as an off-white solid. ¹H NMR (400 MHz, DMSO-d₆) δ 8.08 (s, 1H), 7.9 (broad s, 1H), 7.85 (m, 1H), 7.3-7.5 (complex, 6H), 7.19 (d, J=8.5 Hz, 1H), 5.20 (s, 2H). MS m/z 371.1 (MH⁺).

### EXAMPLE 4

### 4-amino-6-(4-benzyloxy-3-chloro-phenylamino)-pyrimidine-5-carboxylic Acid 2-morpholin-4-yl-ethyl Ester (Cpd 3)

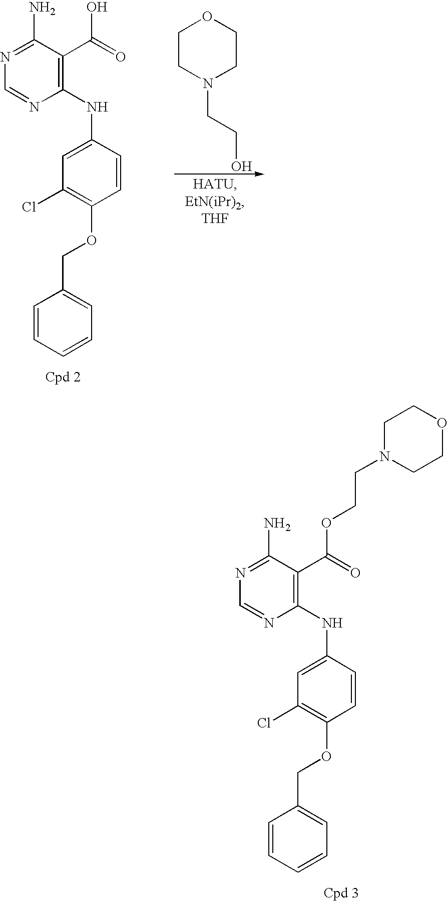

Diisopropylethylamine (59 μL, 44 mg, 0.34 mmol) and HATU (77 mg, 0.20 mmol) were added to a stirring suspension of 50 mg (0.14 mmol) of 4-amino-6-(4-benzyloxy-3-chloro-phenylamino)-pyrimidine-5-carboxylic acid Compound 2 in 0.5 mL of THF. After 30 min, 19 μL (21 mg, 0.16 mmol) of 2-morpholin-4-yl-ethanol was added and the mixture was stirred for 16 h. The mixture was extracted with ethyl acetate and washed with water. The organic layer was concentrated onto silica gel and purified by flash chromatography using 3-7% MeOH/CH₂Cl₂ to yield a yellow solid. The crude product was re-chromatographed using 0-5% MeOH/CH₂Cl₂ to give 18 mg (28%) of 4-amino-6-(4-benzyloxy-3-chloro-phenylamino)-pyrimidine-5-carboxylic acid 2-morpholin-4-yl-ethyl ester Compound 3. ¹H NMR (400 MHz, DMSO-d₆) δ 8.05 (s, 1H), 7.82 (m, 1H), 7.3-7.5 (complex, 5H), 7.19 (m, 1H), 5.20 (s, 2H), 4.40 (m, 2H), 3.53 (m, 4H), 2.68 (m. 2H), 2.44 (m. 4H). MS m/z 482.1 (M-H⁺).

Using the procedure of Example 4, other compounds representative of the present invention were prepared:

| Cpd | Name and Data |
|---|---|
| 4 | 4-amino-6-(4-benzyloxy-3-chloro-phenylamino)-pyrimidine-5-carboxylic acid 3- |
| | fluoro-benzyl ester |
| | ¹H NMR (400 MHz, DMSO-d₆) δ 8.06 (s, 1H), 7.78 (m, 1H), 7.3-7.5 (complex, |
| | 9H), 7.17 (m, 2H), 5.41 (s, 2H), 5.19 (s, 2H). MS m/z 477.0 (M − H⁺) |
| 5 | 4-amino-6-(4-benzyloxy-3-chloro-phenylamino)-pyrimidine-5-carboxylic acid |
| | diethylamide |
| | ¹H NMR (400 MHz, DMSO-d₆) δ 8.02 (s, 2H), 7.65 (d, 1H, J = 2.5 Hz), |
| | 7.3-7.5 (complex, 6H), 7.13 (d, 1H, J = 9 Hz), 6.20 (s, 2H), 5.17 (s, 2H), 3.25 (m, 4H), |
| | 1.07 (m, 6H). MS m/z 426.1 (M + H⁺) |
| 6 | 4-amino-6-(4-benzyloxy-3-chloro-phenylamino)-pyrimidine-5-carboxylic acid (2- |
| | methoxy-ethyl)-amide |
| | ¹H NMR (400 MHz, DMSO-d₆) δ 8.79 (s, 1H), 8.35 (M, 1H), 8.03 (s, 1H), 7.78 (s, |
| | 1H), 7.3-7.5 (complex, 6H), 7.15 (d, 1H, J = 8.8 Hz), 6.62 (s, 2H), 5.17 (s, 2H), |
| | 3.47 (m 2H), 3.40 (m, 2H), 3.26 (s, 3H). MS m/z 427.8 (M + H⁺) |
| 7 | 4-amino-6-(4-benzyloxy-3-chloro-phenylamino)-pyrimidine-5-carboxylic acid (2- |
| | piperidin-1-yl-ethyl)-amide |
| | ¹H NMR (400 MHz, MeOH-d₄) δ 7.96 (s, 2H), 7.59 (s, 1H), 7.46 (m, 2H), |
| | 7.2-7.4 (complex, 4H), 7.07 (d, 1H, J = 9 Hz), 5.16 (s, 2H), 3.53 (t, 2H, J = 6 Hz), 2.52 (t, |
| | 2H, J = 6 Hz), 2.45 (m, 4H), 1.51 (m, 4H), 1.43 (m, 2H). MS m/z 481.2 (M + H⁺) |
| 8 | 4-amino-6-(4-benzyloxy-3-chloro-phenylamino)-pyrimidine-5-carboxylic acid (2- |
| | morpholin-4-yl-ethyl)-amide |
| | ¹H NMR (400 MHz, MeOH-d₄) δ7.97 (s, 1H), 7.59 (d, 1H, J = 2.5 Hz), 7.46 (d, 2H, |
| | J = 7 Hz), 7.37 (t, 2H, J = 7 Hz), 7.3 (m, 1H), 7.23 (dd, 1H, J = 2.5, 9 Hz), 7.07 (d, |
| | 1H, J = 9 Hz), 5.16 (s, 2H), 3.60 (t, 4H, J = 4.5 Hz), 3.55 (t, 2H, J = 6 Hz), 2.57 (t, |
| | 2H, J = 6 Hz), 2.51 (m, 4H). MS m/z 483.1 (M + H⁺) |
| 9 | 4-amino-6-(4-benzyloxy-3-chloro-phenylamino)-pyrimidine-5-carboxylic acid [2- |
| | (4-methoxy-phenyl)-ethyl]-amide |
| | ¹H NMR (400 MHz, DMSO-d₆) δ 8.80 (s, 1H), 8.32 (m, 1H), 8.01 (s, 1H), 7.73 (d, |
| | 1H, J = 2.5 Hz), 7.25-7.45 (complex, 6H), 7.14 (m, 3H), 6.82 (d, 2H, J = 8.5 Hz), |
| | 6.56 (s, 2H), 5.17 (s, 2H), 3.68 (s, 3H), 3.41 (m, 2H), 2.75 (m, 2H). MS m/z |
| | 504.1 (M + H⁺) |
| 10 | 4-amino-6-(4-benzyloxy-3-chloro-phenylamino)-pyrimidine-5-carboxylic acid |
| | phenylamide |
| | ¹H NMR (400 MHz, DMSO-d₆) δ 10.21 (s, 1H), 8.91 (s, 1H), 8.06 (s, 1H), 7.75 (d, |
| | 2H, J = 2.5 Hz), 7.67 (d, 2H, J = 8 Hz), 7.3-7.5 (complex, 9H), 7.12 (d, 1H, J = 9 Hz), |
| | 7.06 (t, 1H, J = 7.5 Hz), 6.76 (s, 2H), 5.15 (s, 2H) MS m/z 446.1 (M + H⁺) |
| 11 | 4-amino-6-(4-benzyloxy-3-chloro-phenylamino)-pyrimidine-5-carboxylic acid (4- |
| | methoxy-phenyl)-amide |
| | ¹H NMR (400 MHz, DMSO-d₆) δ 10.08 (s, 1H), 8.91 (s, 1H), 8.05 (s, 1H), 7.75 (d, |
| | 2H, J = 2.5 Hz), 7.57 (d, 2H, J = 8 Hz), 7.3-7.5 (complex, 8H), 7.12 (d, 1H, J = 9 Hz), |
| | 6.88 (d, 1H, J = 9 Hz), 6.73 (s, 2H), 5.15 (s, 2H), 3.72 (s, 3H). MS m/z |
| | 475.8 (M + H⁺) |

### EXAMPLE 5

### 4-amino-6-(4-benzyloxy-3-chloro-phenylamino)-pyrimidine-5-carboxylic Acid Hydrazide (Cpd 12)

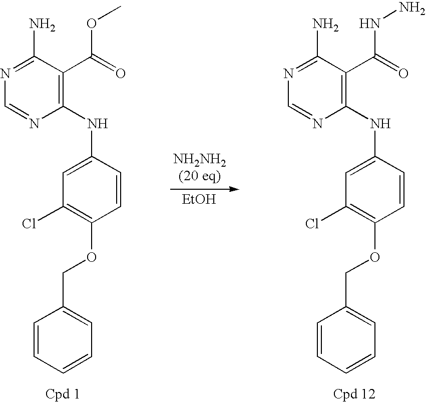

A suspension of Compound 1 (4.3 g, 11.2 mmol) in 100% EtOH (110 mL) was treated with hydrazine (6.8 mL, 224 mol) and warmed at reflux for 24 h. The reaction mixture was concentrated onto SiO₂ (15 g). The residue was purified via column chromatography (Horizon 65+M, 0-10% MeOH/DCM) to provide 4-amino-6-(4-benzyloxy-3-chloro-phenylamino)-pyrimidine-5-carboxylic acid hydrazide Compound 12 (2.50 g, 58%) as a yellow solid. ¹H NMR (300 MHz, CD₃OD) δ 7.96 (s, 1H), 7.61 (d, J=2.0 Hz, 1H), 7.64-7.22 (m, 6H), 7.04 (d, J=8.7 Hz, 1H), 5.22 (s, 2H); MS m/z 385.0 (MH⁺).

### EXAMPLE 6

### 4-amino-6-(4-benzyloxy-3-chloro-phenylamino)-pyrimidine-5-carboxylic Acid N′-(3-morpholin-4-yl-propionyl)-hydrazide (Cpd 13)

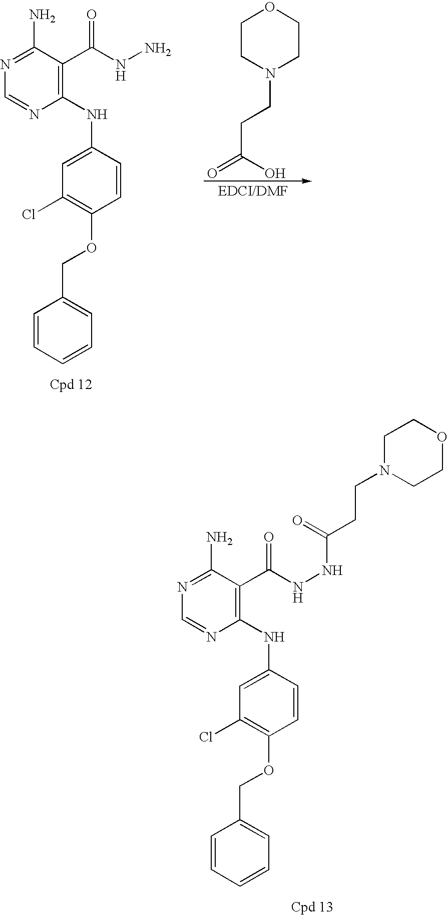

A mixture of Compound 12 (6.8 mg, 0.018 mmol), 3-morpholin-4-yl-propionic acid hydrochloride (3.8 mg, 0.019 mmol) and EDCI (10.2 mg, 0.053 mmol) in DMF (0.17 mL) was stirred at rt for 12 h. The reaction was diluted with 1120 (20 mL) and extracted with EtOAc (3×10 mL). The combined organic extracts were dried (Na₂SO₄) and concentrated to afford 4-amino-6-(4-benzyloxy-3-chloro-phenylamino)-pyrimidine-5-carboxylic acid N′-(3-morpholin-4-yl-propionyl)-hydrazide Compound 13 (8.3 mg, 88%) as a yellow solid. ¹H NMR (400 MHz, CD₃OD) δ 8.03 (s, 1H), 7.76 (d, J=1.2 Hz, 1H), 7.47-7.30 (m, 6H), 7.05 (d, J=6.4 Hz, 1H), 5.15 (s, 2H), 2.76 (m, 2H), 2.56 (m, 6H), 1.29 (br s, 6H); MS m/z 526.5 (MH⁺).

### BIOLOGICAL EXAMPLES

The ability of the compounds for treating, preventing or ameliorating a chronic or acute kinase mediated disease, disorder or condition was determined using the following procedures.

### Example 1

### EGFR Kinase Assay

The EGFR kinase used was a fusion of Glutathione-S-Transferase (GST) and a PCR amplified intracellular portion of EGFR (NM_{—}005228). The intracellular portion of EGFR started at nucleotide 2189 (corresponding to amino acid 667) and ended at the termination codon. The portion was PCR amplified with primers that added the lambda attB sequences to each end, recombined into an entry vector, then into a (GST destination vector (as described in Gateway Technologies Manual by Invitrogen Corporation, Carlsbad, Calif.).

The destination vector was recombined in the DH10BAC strain of bacteria to produce a bacmid. The bacmid was transfected into Sf 9 cells and the supernatant containing the baculovirus was collected. The GSTEGFR protein was purified using large cultures of Sf 9 cells infected with stock virus. After an appropriate period of time, the cells were collected and lysed. The GSTEGFR was then purified from the lysate on Glutathione-Sepharose columns (as described by Amersham Biosciences, Buckinghamshire, United Kingdom).

The EGFR substrate was prepared by biotinylating polyGluTyr (128 mg) (Sigma, St. Louis, Mo.) in a 1×PBS buffer incubated together with a 12-fold molar excess of Sulfo-NHS-LC-Biotin on ice for at least 2 hrs. The free biotin was separated from the biotinylated polyGluTyr on a gel filtration column.

A mixture of a 10× kinase buffer (500 mM Tris at pH 8.0, 100 mM Magnesium Chloride and 1 mM Sodium Vanadate), DTT (1 mM final from 500 mM stock), ATP (5 μM final from 10 nm stock), biotinylated polyGluTyr (10 μg/μL stock), γ-³³P ATP (10 μCi/μL stock) and water was added to each well (90 μL/well) of a Streptavidin Flashplate (Perkin Elmer, Wellesley, Mass.).

Test compound in 100% DMSO (2 μL) was added to the appropriate wells. Diluted GSTEGFR (1:300 dilution in 50 mM Tris at pH 8.0 and 0.1% bovine serum albumin) (10 μL) was added to the wells to initiate the reactions.

The plates were incubated at 30° C. for 1 hr with shaking, The reacted contents were removed and the plates were sequentially washed three times with a 1×PBS stop buffer (300 μL without Magnesium and Calcium) and 100 mM EDTA. After the final wash, the same stop buffer (200 μL) was added to the wells. The plates were then sealed and read on the TopCount scintillation counter.

Test compounds were assayed in triplicate at 16 concentrations at half-log dilutions starting at 200 μM. A maximum and minimum signal for the assay was determined on each plate. The percent inhibition of a test compound was calculated according to the formula

$$[ \frac{( max signal - test compound )}{( max signal - min signal )} ] ( 100 ) = \% inhibition$$

For a series of test concentrations, the IC₅₀ was derived by graphing percent inhibition against the log of the concentrations tested for a given compound. The ECFR IC₅₀ results are shown in Table 1. Values shown as % indicate % inhibition at a test concentration of 2 μM.

TABLE 1
EGFR IC₅₀ (μM)

| Cpd | IC₅₀ |
|---|---|
| 1 | 0.054 |
| 2 | 0.042 |
| 3 | 0.16 |
| 4 | 34% |
| 5 | 36% |
| 6 | 37% |
| 7 | 42% |
| 8 | 35% |
| 9 | 28% |
| 10 | 52% |
| 11 | 36% |
| 12 | 0.46 |
| 13 | 0.62 |
| 14 | 0.076 |

### Example 2

### HER-2 Kinase Assay

The HER-2 kinase used was purified at Proqinase (Freiburg, Germany) from a construct that consisted of a fusion of GST (Glutathione-S-Transferase), HIS6-Thrombin and the nucleotides encoding amino acids 679 to 1255 of HER-2 (Accession Number M11730).

A mixture of a 10× kinase reaction buffer (600 mM Hepes at pH 7.5, 30 mM Magnesium Chloride, 0.03 mM Sodium Vanadate and 500 μg/mL PEG 20,000), DTT (1.2 mM final from a 10 mM stock), ATP (1 μM from a 10 mM stock), biotinylated polyGluTyr (1.5 ng/μL final from stock of 1 μg/μL) prepared by Upstate Biotechnologies, Lake Placid, N.Y.), Manganese Chloride (3 mM final from a 1 M stock), γ-³³P-ATP (10 μCi/μL stock) and water (70 μL/well) was added to each well of a Streptavidin Flashplate (Cat. # SMP103, NEN, Boston, Mass.).

Test compound stock (1 μL) was added to the appropriate wells. Diluted GSTHER2 kinase (6.7 ng/μL diluted into 50 mM Tris-HCl at pH 8.0 and 0.1% bovine serum albumin) (30 μL) was added (total volume of 200 ng/well) to initiate the reactions.

The reaction plates were incubated at 30° C. for 1 hr. The reaction was terminated by aspirating the reaction mixture from the plate wells and washing the wells three times with a 1× PBS stop buffer (300 μL) and 100 mM EDTA. After the final wash, the same stop buffer (200 μL) was again added to the wells. The plates were then sealed and read on the TopCount scintillation counter.

Test compounds were assayed in triplicate at 8 concentrations at one-log dilutions starting at 100 μM. A maximum and minimum signal for the assay was determined on each plate. The percent inhibition of a test compound was calculated according to the formula:

$$[ \frac{( max signal - test compound )}{( max signal - min signal )} ] ( 100 ) = \% inhibition$$

For a series of test concentrations, the IC₅₀ was derived by graphing percent inhibition against the log of the concentrations tested for a given compound. The Her-2 IC₅₀ values are shown in Table 2. Values shown as % indicate % inhibition at a test concentration of 100 μM.

TABLE 2
HER2 IC₅₀ (μM)

| Cpd | IC₅₀ |
|---|---|
| 1 | 92% |
| 2 | 0.78 |
| 3 | 1.38 |
| 4 | 21% |
| 5 | 39% |
| 6 | 33% |
| 7 | 10.0 |
| 8 | 82% |
| 9 | 4% |
| 10 | 12% |
| 11 | 0% |
| 12 | 0.15 |
| 13 | 5.7 |
| 14 | 63% |

### Assay to Measure Inhibition of In Vitro Cell Proliferation

The ability of a test compound to inhibit the proliferation of cell growth was determined by measuring incorporation of ¹⁴C-labelled thymidine into newly synthesized DNA within the cells. This method was used on American Type Culture Collection (ATCC, Virginia) cell lines derived from carcinomas originating from several tissues such as HeLa cervical adenocarcinoma (ATCC Cat. #CCL-2), HCT-116 colon carcinoma (ATCC Cat. #CCL-247), and A375 malignant melanoma (ATCC Cat. #CRL-1619).

The carcinoma cells are trypsinized and counted. The cells (3000-8000 count) are added to each well of a 96-well CytoStar tissue culture treated scintillating microplate (Amersham #RPNQ0160) in complete medium (100 μL) and the plate is then incubated in complete medium for 24 hrs at 37° C. in an inert atmosphere containing 5% CO₂.

Test compound (1 μL) in 100% DMSO is added to the plate test-wells with DMSO only added to control-wells. The plate is incubated in complete medium for a second 24 hr period at 37° C. in an atmosphere containing 5% CO₂.

An aliquot of a solution of Methyl ¹⁴C-thymidine (56 mC/mmol) (NEN #NEC568 or Amersham #CFA532) in complete medium (20 uL to provide 0.2 μCi/well) is then added to each well and the plate is incubated for a third 24 hr period at 37° C. in an atmosphere containing 5% CO₂.

The plate contents are then discarded, the plate is washed twice with PBS (200 μL) and then PBS (200 μL) is added to each well. The plate is sealed and the degree of methyl ¹⁴C-thymidine incorporation is quantified on a Packard Top Count.

The IC₅₀ values for the compounds tested in various cell lines are shown in Table 3.

TABLE 3

| | IC₅₀ (μM) | IC₅₀ (μM) | IC₅₀ (μM) |
|---|---|---|---|
| Cpd | HeLa | HCT116 | A375 |
| 1 | 1.9 | 3.0 | 1 |
| 2 | >10 | >10 | >10 |
| 3 | >10 | 16 | 5.1 |
| 4 | >10 | >10 | >10 |
| 5 | >10 | >10 | >10 |
| 6 | >100 | >100 | >100 |
| 7 | 34 | >10 | >1 |
| 8 | >10 | >10 | >10 |
| 9 | >100 | >100 | >100 |
| 10 | >100 | >100 | >100 |
| 11 | >100 | >100 | >100 |
| 12 | 3.0 | 3.4 | 1.7 |
| 13 | >100 | >100 | >100 |

While the foregoing specification teaches the principles of the present invention, with examples provided for the purpose of illustration, it will be understood that the practice of the invention encompasses all of the usual variations, adaptations and modifications as come within the scope of the following claims and their equivalents.

Throughout this application, various publications are cited. These publications are hereby incorporated by reference in their entirety into this application to describe more fully the state of the art to which this invention pertains.

What is claimed is:

1. A compound of Formula (I) 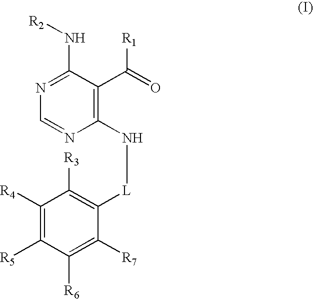
and forms thereof, wherein
L is selected from the group consisting of a bond, C₁₋₆alkyl and halo-C₁₋₆alkyl;
R₁ is selected from the group consisting of hydrogen, hydroxy, C₁₋₈alkoxy, hydroxy-C₁₋₈alkoxy, amino, C₁₋₈alkyl-amino, C₁₋₈alkoxy-C₁₋₈alkyl-amino, amino-amino, C₁₋₈alkyl-amino-amino, C₁₋₈alkyl-carbonyl-amino-amino, aryl-C₁₋₈alkoxy, aryl-amino, aryl-C₁₋₈alkyl-amino, heterocyclyl-C₁₋₈alkoxy, heterocyclyl-C₁₋₈alkyl-amino, heterocyclyl-C₁₋₈alkyl-carbonyl-amino, heterocyclyl-C₁₋₈alkyl-carbonyl-amino-amino, heterocyclyl and benzofused-heterocyclyl,
wherein each instance of aryl is optionally substituted with one, two, three, four or five substituents each selected from the group consisting of halogen, hydroxy, C₁₋₈alkyl, C₁₋₈alkoxy, amino, C₁₋₈alkyl-amino, C₁₋₈alkyl-carbonyl and C₁₋₈alkoxy-carbonyl, and
wherein each instance of heterocyclyl is optionally substituted with one, two, three or four substituents each selected from the group consisting of hydroxy, C₁₋₈alkyl, C₁₋₈alkoxy, amino, C₁₋₈alkyl-amino, C₁₋₈alkyl-carbonyl and C₁₋₈alkoxy-carbonyl;
R₂ is selected from the group consisting of hydrogen, C₁₋₈alkyl and C₁₋₈alkoxy; and
R₃, R₄, R₅, R₆ and R₇ are each selected from the group consisting of hydrogen, halogen, hydroxy, cyano, nitro, C₁₋₈alkyl, C₁₋₈alkoxy, C₁₋₈alkoxy-C₁₋₈alkyl, hydroxy-C₁₋₈alkyl, halo-C₁₋₈alkyl, hydroxy-C₁₋₈alkoxy, halo-C₁₋₈alkoxy, cyano-C₁₋₈alkyl, amino, C₁₋₈alkyl-amino, amino-C₁₋₈alkyl, C₁₋₈alkyl-amino-C₁₋₈alkyl, C₁₋₈alkyl-amino-C₁₋₈alkyl-amino-carbonyl, C₁₋₈alkoxy-imino-C₈alkyl, C₁₋₈alkoxy-imino-(aryl)C₁₋₈alkyl, carboxy, C₁₋₈acyl, C₁₋₈acyl-amino, C₁₋₈alkyl-carbonyl, C₁₋₈alkoxy-carbonyl, thio-C₁₋₈alkyl, substituted phosphonic acid, C₃₋₁₂cycloalkyl, aryl, aryloxy, aryl-amino, aryl-C₁₋₈alkyl, aryl-C₁₋₈alkoxy, aryl-carbonyl-C₁₋₈alkyl, aryl-amido, heteroaryl, heteroaryloxy, heteroaryl-C₁₁ alkoxy, heteroaryl-amino-sulfonyl, heterocyclyl and benzofused-heterocyclyl,
wherein phosphonic acid is substituted on the phosphorous atom with two substituents selected from the group consisting of hydroxy and C₁₋₈alkoxy,
wherein each instance of heterocyclyl and benzofused-heterocyclyl is optionally substituted on one or two heterocyclyl carbon atoms with an oxo substituent, and
wherein each instance of heteroaryl is optionally substituted with one, two, three, four or five substituents each selected from the group consisting of C₁₋₈alkyl, amino-C₁₋₈alkyl, C₁₋₈alkyl-amino-C₁₋₈alkyl, carboxy, C₁₋₈acyl and C₁₋₈alkoxy-carbonyl.

2. The compound of claim 1, wherein
L is a bond;
R₁ is selected from the group consisting of hydrogen, hydroxy, C₁₋₈alkoxy, C₁₋₈alkyl-amino, C₁₋₈alkoxy-C₁₋₈alkyl-amino, amino-amino, aryl-C₁₋₈alkoxy, aryl-amino, aryl-C₁₋₈alkyl-amino, heterocyclyl-C₁₋₈alkoxy, heterocyclyl-C₁₋₈alkyl-amino, and heterocyclyl-C₁₋₈alkyl-carbonyl-amino-amino,
wherein each instance of aryl is optionally substituted with one substituent selected from the group consisting of halogen and C₁₋₈alkoxy;
R₂ is hydrogen; and
R₃, R₄, R₅, R₆ and R₇ are each selected from the group consisting of hydrogen, halogen, and aryl-C₁₋₈alkoxy.

3. The compound of claim 1, wherein said compound has the following Formula (Ia): 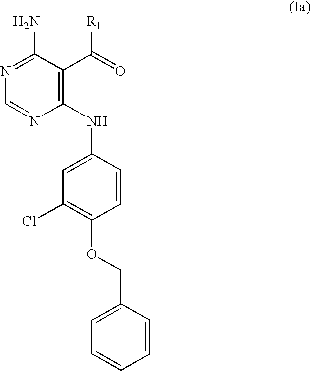
and forms thereof, wherein
R₁ is selected from the group consisting of hydrogen, hydroxy, C₁₋₈alkoxy, C₁₋₈alkyl-amino, C₁₋₈alkoxy-C₁₋₈alkyl-amino, amino-amino, aryl-C₁₋₈alkoxy, aryl-amino, aryl-C₁₋₈alkyl-amino, heterocyclyl-C₁₋₈alkoxy, heterocyclyl-C₁₋₈alkyl-amino, and heterocyclyl-C₁₋₈alkyl-carbonyl-amino-amino,
wherein each instance of aryl is optionally substituted with one substituent selected from the group consisting of halogen and C₁₋₈alkoxy.

4. The compound of claim 3, wherein
R₁ is selected from the group consisting of hydrogen, hydroxy, C₁₋₈alkoxy, amino-amino, aryl-amino, heterocyclyl-C₁₋₈alkoxy, heterocyclyl-C₁₋₈alkyl-amino, and heterocyclyl-C₁₋₈alkyl-carbonyl-amino-amino,
wherein aryl is optionally substituted with one substituent selected from the group consisting of halogen and C₁₋₈alkoxy.

5. The compound of claim 1, wherein said compound is selected from the group consisting of:
4-amino-6-(4-benzyloxy-3-chloro-phenylamino)-pyrimidine-5-carboxylic acid methyl ester,
4-amino-6-(4-benzyloxy-3-chloro-phenylamino)-pyrimidine-5-carboxylic acid,
4-amino-6-(4-benzyloxy-3-chloro-phenylamino)-pyrimidine-5-carboxylic acid 2-morpholin-4-yl-ethyl ester,
4-amino-6-(4-benzyloxy-3-chloro-phenylamino)-pyrimidine-5-carboxylic acid 3-fluoro-benzyl ester,
4-amino-6-(4-benzyloxy-3-chloro-phenylamino)-pyrimidine-5-carboxylic acid diethylamide,
4-amino-6-(4-benzyloxy-3-chloro-phenylamino)-pyrimidine-5-carboxylic acid (2-methoxy-ethyl)-amide,
4-amino-6-(4-benzyloxy-3-chloro-phenylamino)-pyrimidine-5-carboxylic acid (2-piperidin-1-yl-ethyl)-amide,
4-amino-6-(4-benzyloxy-3-chloro-phenylamino)-pyrimidine-5-carboxylic acid (2-morpholin-4-yl-ethyl)-amide,
4-amino-6-(4-benzyloxy-3-chloro-phenylamino)-pyrimidine-5-carboxylic acid [2-(4-methoxy-phenyl)-ethyl]-amide,
4-amino-6-(4-benzyloxy-3-chloro-phenylamino)-pyrimidine-5-carboxylic acid phenylamide, and
4-amino-6-(4-benzyloxy-3-chloro-phenylamino)-pyrimidine-5-carboxylic acid (4-methoxy-phenyl)-amide.

6. A method of inhibiting a protein kinase comprising administering a compound of Formula (I) 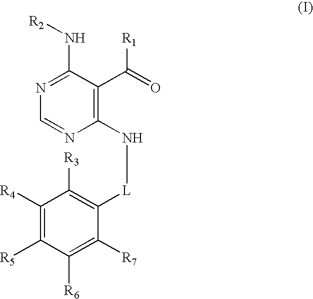
and forms thereof, wherein
L is selected from the group consisting of a bond, C₁₋₆alkyl and halo-C₁₋₆alkyl;
R₁ is selected from the group consisting of hydrogen, hydroxy, C₁₋₆alkoxy, hydroxy-C₁₋₈alkoxy, amino, C₁₋₈alkyl-amino, C₁₋₈alkoxy-C₁₋₈alkyl-amino, amino-amino, C₁₋₈alkyl-amino-amino, C₁₋₈alkyl-carbonyl-amino-amino, aryl-C₁₋₈alkoxy, aryl-amino, aryl-C₁₋₈alkyl-amino, heterocyclyl-C₁₋₈alkoxy, heterocyclyl-C₁₋₈alkyl-amino, heterocyclyl-C₁₋₈alkyl-carbonyl-amino, heterocyclyl-C₁₋₈alkyl-carbonyl-amino-amino, heterocyclyl and benzofused-heterocyclyl,
wherein each instance of aryl is optionally substituted with one, two, three, four or five substituents each selected from the group consisting of halogen, hydroxy, C₁₋₈alkyl, C₁₋₈alkoxy, amino, C₁₋₈alkyl-amino, C₁₋₈alkyl-carbonyl and C₁₋₈alkoxy-carbonyl, and
wherein each instance of heterocyclyl is optionally substituted with one, two, three or four substituents each selected from the group consisting of hydroxy, C₁₋₈alkyl, C₁₋₈alkoxy, amino, C₁₋₈alkyl-amino, C₁₋₈alkyl-carbonyl and C₁₋₈alkoxy-carbonyl;
R₂ is selected from the group consisting of hydrogen, C₁₋₈alkyl and C₁₋₈alkoxy; and
R₃, R₄, R₅, R₆ and R₇ are each selected from the group consisting of hydrogen, halogen, hydroxy, cyano, nitro, C₁₋₈alkyl, C₁₋₈alkoxy, C₁₋₈alkoxy-C₁₋₈alkyl, hydroxy-C₁₋₈alkyl, halo-C₁₋₈alkyl, hydroxy-C₁₋₈alkoxy, halo-C₁₋₈alkoxy, cyano-C₁₋₈alkyl, amino, C₁₋₈alkyl-amino, amino-C₁₋₈alkyl, C₁₋₈alkyl-amino-C₁₋₈alkyl, C₁₋₈alkyl-amino-C₁₋₈alkyl-amino-carbonyl, C₁₋₈alkoxy-imino-C₁₋₈alkyl, C₁₋₈alkoxy-imino-(aryl)C₁₋₈alkyl, carboxy, C₁₋₈acyl, C₁₋₈acyl-amino, C₁₋₈alkyl-carbonyl, C₁₋₈alkoxy-carbonyl, thio-C₁₋₈alkyl, substituted phosphonic acid, C₁₋₈cycloalkyl, aryl, aryloxy, aryl-amino, aryl-C₁₋₈alkyl, aryl-C₁₋₈alkoxy, aryl-carbonyl-C₁₋₈alkyl, aryl-amido, heteroaryl, heteroaryloxy, heteroaryl-C₁₋₈alkoxy, heteroaryl-amino-sulfonyl, heterocyclyl and benzofused-heterocyclyl,
wherein phosphonic acid is substituted on the phosphorous atom with two substituents selected from the group consisting of hydroxy and C₁₋₈alkoxy,
wherein each instance of heterocyclyl and benzofused-heterocyclyl is optionally substituted on one or two heterocyclyl carbon atoms with an oxo substituent, and
wherein each instance of heteroaryl is optionally substituted with one, two, three, four or five substituents each selected from the group consisting of C₁₋₈alkyl, amino-C₁₋₈alkyl, C₁₋₈alkyl-amino-C₁₋₈alkyl, carboxy, C₁₋₈acyl and C₁₋₈alkoxy-carbonyl.

7. The method of claim 6, wherein
L is a bond;
R₁ is selected from the group consisting of hydrogen, hydroxy, C₁₋₈alkoxy, C₁₋₈alkyl-amino, C₁₋₈alkoxy-C₁₋₈alkyl-amino, amino-amino, aryl-C₁₋₈alkoxy, aryl-amino, aryl-C₁₋₈alkyl-amino, heterocyclyl-C₁₋₈alkoxy, heterocyclyl-C₁₋₈alkyl-amino, and heterocyclyl-C₁₋₈alkyl-carbonyl-amino-amino,
wherein each instance of aryl is optionally substituted with one substituent selected from the group consisting of halogen and C₁₋₈alkoxy;
R₂ is hydrogen; and
R₃, R₄, R₅, R₆ and R₇ are each selected from the group consisting of hydrogen, halogen, and aryl-C₁₋₈alkoxy.

8. The method of claim 6, comprising administering a compound Formula (Ia): 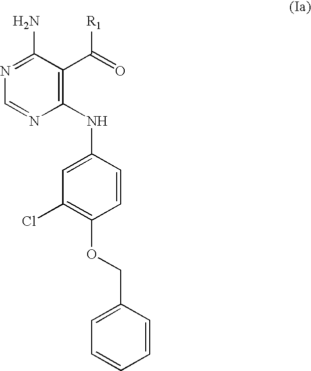
and forms thereof, wherein
R₁ is selected from the group consisting of hydrogen, hydroxy, C₁₋₈alkoxy, C₁₋₈alkyl-amino, C₁₋₈alkoxy-C₁₋₈alkyl-amino, amino-amino, aryl-C₁₋₈alkoxy, aryl-amino, aryl-C₁₋₈alkyl-amino, heterocyclyl-C₁₋₈alkoxy, heterocyclyl-C₁₋₈alkyl-amino, and heterocyclyl-C₁₋₈alkyl-carbonyl-amino-amino,
wherein each instance of aryl is optionally substituted with one substituent selected from the group consisting of halogen and C₁₋₈alkoxy.

9. The method of claim 8, comprising administering compound of Formula (Ia),
wherein
R₁ is selected from the group consisting of hydrogen, hydroxy, C₁₋₈alkoxy, amino-amino, aryl-amino, heterocyclyl-C₁₋₈alkoxy, heterocyclyl-C₁₋₈alkyl-amino, and heterocyclyl-C₁₋₈alkyl-carbonyl-amino-amino,
wherein aryl is optionally substituted with one substituent selected from the group consisting of halogen and C₁₋₈alkoxy.

10. The method of claim 6, wherein said compound is selected from the group consisting of:
4-amino-6-(4-benzyloxy-3-chloro-phenylamino)-pyrimidine-5-carboxylic acid methyl ester,
4-amino-6-(4-benzyloxy-3-chloro-phenylamino)-pyrimidine-5-carboxylic acid,
4-amino-6-(4-benzyloxy-3-chloro-phenylamino)-pyrimidine-5-carboxylic acid 2-morpholin-4-yl-ethyl ester,
4-amino-6-(4-benzyloxy-3-chloro-phenylamino)-pyrimidine-5-carboxylic acid 3-fluoro-benzyl ester,
4-amino-6-(4-benzyloxy-3-chloro-phenylamino)-pyrimidine-5-carboxylic acid diethylamide,
4-amino-6-(4-benzyloxy-3-chloro-phenylamino)-pyrimidine-5-carboxylic acid (2-methoxy-ethyl)-amide,
4-amino-6-(4-benzyloxy-3-chloro-phenylamino)-pyrimidine-1-carboxylic acid (2-piperidin-1-yl-ethyl)-amide,
4-amino-6-(4-benzyloxy-3-chloro-phenylamino)-pyrimidine-5-carboxylic acid (2-morpholin-4-yl-ethyl)-amide,
4-amino-6-(4-benzyloxy-3-chloro-phenylamino)-pyrimidine-5-carboxylic acid [2-(4-methoxy-phenyl)-ethyl]-amide,
4-amino-6-(4-benzyloxy-3-chloro-phenylamino)-pyrimidine-5-carboxylic acid phenylamide, and
4-amino-6-(4-benzyloxy-3-chloro-phenylamino)-pyrimidine-5-carboxylic acid (4-methoxy-phenyl)-amide.

11. A method of inhibiting a protein kinase comprising administering a compound selected from the group consisting of 4-amino-6-(4-benzyloxy-3-chloro-phenylamino)-pyrimidine-5-carboxaldehyde; amino-6-(4-benzyloxy-3-chloro-phenylamino)-pyrimidine-5-carboxylic acid hydrazide; and 4-amino-6-(4-benzyloxy-3-chloro-phenylamino)-pyrimidine-5-carboxylic acid N′-(3-morpholin-4-yl-propionyl)-hydrazide.

12. The method of claim 6, wherein said method is employed to treat, prevent or ameliorate a disease, disorder or condition selected from the group consisting of osteoarthritis, rheumatoid arthritis, synovial pannus invasion in arthritis, multiple sclerosis, myasthenia gravis, diabetes mellitus, diabetic angiopathy, diabetic retinopathy, retinal vessel proliferation, inflammatory bowel disease, Crohn's disease, ulcerative colitis, bone diseases, transplant or bone marrow transplant rejection, lupus, chronic pancreatitis, cachexia, septic shock, fibroproliferative and differentiative skin disease or disorder, central nervous system disease, neurodegenerative disease, disorder or condition related to nerve damage and axon degeneration subsequent to a brain or spinal cord injury, acute or chronic cancer, ocular diseases, viral infection, heart disease, lung or pulmonary disease or kidney or renal disease.

13. The method of claim 11, wherein said method is employed to treat, prevent or ameliorate a disease, disorder or condition selected from the group consisting of osteoarthritis, rheumatoid arthritis, synovial pannus invasion in arthritis, multiple sclerosis, myasthenia gravis, diabetes mellitus, diabetic angiopathy, diabetic retinopathy, retinal vessel proliferation, inflammatory bowel disease, Crohn's disease, ulcerative colitis, bone diseases, transplant or bone marrow transplant rejection, lupus, chronic pancreatitis, cachexia, septic shock, fibroproliferative and differentiative skin disease or disorder, central nervous system disease, neurodegenerative disease, disorder or condition related to nerve damage and axon degeneration subsequent to a brain or spinal cord injury, acute or chronic cancer, ocular diseases, viral infection, heart disease, lung or pulmonary disease or kidney or renal disease.
